(19) United States
(12) Patent Application Publication
(10) Pub. No.: US 2022/0230714 A1
(43) Pub. Date: Jul. 21, 2022

(54) DASHBOARDS FOR CLINICAL WORKFLOW AND PATIENT HANDOFF ASSISTANCE

(71) Applicant: Hill-Rom Services, Inc., Batesville, IN (US)

(72) Inventors: Sinan Batman, Cary, NC (US); Dana E. Peco, Bradenton, FL (US); Eugene G. Urrutia, Apex, NC (US); Karrie Ann Schwencer, Cary, NC (US); Neal E. Wiggermann, Batesville, IN (US); Lori A. Zapfe, Milroy, IN (US)

(21) Appl. No.: 17/578,318

(22) Filed: Jan. 18, 2022

Related U.S. Application Data
(60) Provisional application No. 63/139,280, filed on Jan. 19, 2021.

(51) Int. Cl. G16H 10/20; G16H 40/67; G16H 50/50

(52) U.S. Cl. CPC G16H 10/20; G16H 50/50; G16H 40/67

(57) ABSTRACT

Various techniques for assisting care providers with workflows and handoffs are described. An example method includes identifying patient data that includes message data, electronic medical record (EMR) data, and sensor data. The message data includes at least one text, audio, or video message about a patient. The EMR data includes at least a portion of an EMR of the patient. The sensor data includes at least one image the patient detected by a camera mounted on a hospital bed on which the patient is supported. The example method further includes determining, based on the patient data, a condition of the patient and a task associated with the patient. A report including the condition of the patient and the task is generated and transmitted to a computing device associated with the care provider.

### PRIORITY

This application claims the benefit of priority to U.S. Provisional Patent Application No. 63/139,280, filed on Jan. 19, 2021, and is fully incorporated by reference herein.

### TECHNICAL FIELD

This disclosure relates generally to the technical field of medical devices and electronic platforms to assist caregivers with clinical tasks.

### BACKGROUND

In clinical environments, a single care provider (e.g., a nurse or a physician) may be responsible for caring for multiple patients during a workday or shift. The care provider may be responsible for monitoring the statuses of the patients over time. In some cases, the care provider may be responsible for treating the patients during the workday or shift, such as administering medications, performing physical therapy, or changing wound dressings. The care provider may be responsible for other activities related to the patient, such as changing sheets on a hospital bed or providing information to family members of the patient. Accordingly, the care provider may perform various tasks associated with the patients during the workday or shift.

To perform these tasks, the care provider may move between different parts of the clinical environment. For example, the care provider may take one patient's vital signs in one room on one floor of the clinical environment, and then take another patient's vital signs in another room on another floor of the clinical environment. In addition, the care provider may travel through the clinical environment to acquire equipment for performing various tasks associated with the patients. For instance, the care provider may acquire a gurney from an equipment room in the clinical environment, take the gurney to a patient's room, and use the gurney to move the patient to a radiology wing for scheduled imaging. However, when care providers are responsible for caring for multiple patients simultaneously, they may have limited time to travel through the clinical environment.

When the care provider finishes the workday or shift (or takes a break from caring from the patients), another care provider may be responsible for continuing the care of the patients. The first care provider may prepare a report or discuss the status of the patients with the next care provider. Ideally, the next care provider has all of the information they need to seamlessly care for the patients during their workday or shift. However, in practice, key patient information can be lost as the patients are transferred between care providers.

### SUMMARY

The present disclosure addresses several problems associated with care provider workflow management. In various examples, care providers spend a significant amount of their workday or shift traveling between locations within a clinical environment and/or obtaining equipment to care for patients in the clinical environment. In particular, care providers often spend time initiating tasks that they cannot complete. For example, a care provider seeking to take vital signs of a patient may obtain equipment and travel to a room of the patient only to find that the patient is sleeping, in an appoint with another care provider, or is eating. Care providers could save a significant amount of time in their schedules if they are aware, in advance of visiting patients, that the patients are available.

Various implementations described herein relate to providing assistance to care providers managing multiple patients in a clinical environment. In some cases, a workflow system provides a workflow report to a clinical device associated with a care provider. The workflow report summarizes tasks and/or statuses of various patients being cared for by the care provider. In various examples, the workflow system generates and/or updates the workflow report based on various patient data associated with the patients. In some examples, the patient data is accessed from electronic medical records (EMRs) of the patients. According to some implementations, the patient data is obtained from sensors (e.g., cameras, load sensors, physiological parameter sensors, etc.) monitoring the patients in real-time. The patient data may include, in some cases, messages transmitted between devices associated with care providers, the patients, and patient family members about the patients.

According to some implementations, the workflow system tracks and/or predicts whether patients are ready for tasks to be completed. For example, the workflow system may determine, based on the patient data, that a patient is sleeping and indicate, in the workflow report, that a task associated with the patient is not currently completable. In some cases, the workflow system may predict, based on the patient data, when a patient will be awake and indicate, in the workflow report, that a task associated with the patient can be completed at a time when the patient is predicted to be awake. Thus, care providers can minimize time in their shifts or workdays spent preparing for tasks that cannot be completed.

In some cases, the workflow system generates and/or updates the workflow report by predicting clinically relevant risks and/or events associated with the patients. For example, the workflow system may be configured to determine, based on the patient data, a fall risk, sepsis risk, or other type of risk associated with an individual patient, and may prioritize the individual patient over other patients based on the risk.

In some implementations, the workflow report indicates a priority of patients and/or tasks based on the patient data. The workflow report may indicate the priority of the individuals graphically or via some other user interface technique. In various implementations, the workflow report includes a task-based view that arranges tasks associated with one or more of the patients by urgency and/or importance. The workflow report assists care providers with efficiently completing tasks associated with the neediest patients in the clinical environment.

According to various implementations, the workflow system is configured to monitor patient data associated with patients being cared for by a first care provider and to generate, based on the patient data, a handoff report for a second care provider taking over care of the patients. In various cases, the workflow system determines what data, among a voluminous amount of possible patient data, is relevant to the second care provider. The workflow system may generate the handoff report based on a default template associated with the clinical environment or a personalized template associated with the second care provider. For example, the care provider may prefer the handoff report to include numerous details about the patients, whereas other care providers may prefer handoff reports to include minimal details about the patients. In some cases, the workflow system learns what types of details are most important to the second care provider and generates the handoff report to include those types of details. In some examples, the workflow system may prompt the first care provider to enter and/or record critical handoff information for the benefit of the second care provider and include that information in the handoff report.

In various examples, the workflow system further provides the second care provider with on-demand assistance during the shift or workday of the second care provider. For example, the second care provider may input inquiries about any of the patients being managed by the second care provider into a clinical device associated with the second care provider, which may forward the inquiries to the handoff system. The workflow system may generate answers to the inquiries based on the patient data and may provide the answers to the clinical device. Thus, if the second care provider requests information about the patients beyond what is summarized in the handoff report, the handoff system can provide the requested information.

### DESCRIPTION OF THE FIGURES

The following figures, which form a part of this disclosure, are illustrative of described technology and are not meant to limit the scope of the claims in any manner.

FIG. 1 illustrates an example networking environment for providing assistance to care providers in a clinical environment.

FIG. 2 illustrates example signaling for providing a workflow report to a clinical device.

FIG. 3 illustrates an example graphical user interface (GUI) of a workflow report in a patient mode.

FIG. 4 illustrates an example GUI of a workflow report in a task mode.

FIG. 5 illustrates example signaling for providing a handoff report to the second clinical device.

FIG. 6 illustrates an example GUI of a handoff report output by a clinical device.

FIG. 7 illustrates example signaling for optimizing a handoff report for a particular care provider.

FIG. 8 illustrates an example process for assisting a care provider by providing a report summarizing current and/or potential conditions of a patient assigned to the care provider.

FIG. 9 illustrates at least one example device configured to enable and/or perform the some or all of the functionality discussed herein.

### DETAILED DESCRIPTION

Various implementations of the present disclosure will be described in detail with reference to the drawings, wherein like reference numerals present like parts and assemblies throughout the several views. Additionally, any samples set forth in this specification are not intended to be limiting and merely set forth some of the many possible implementations.

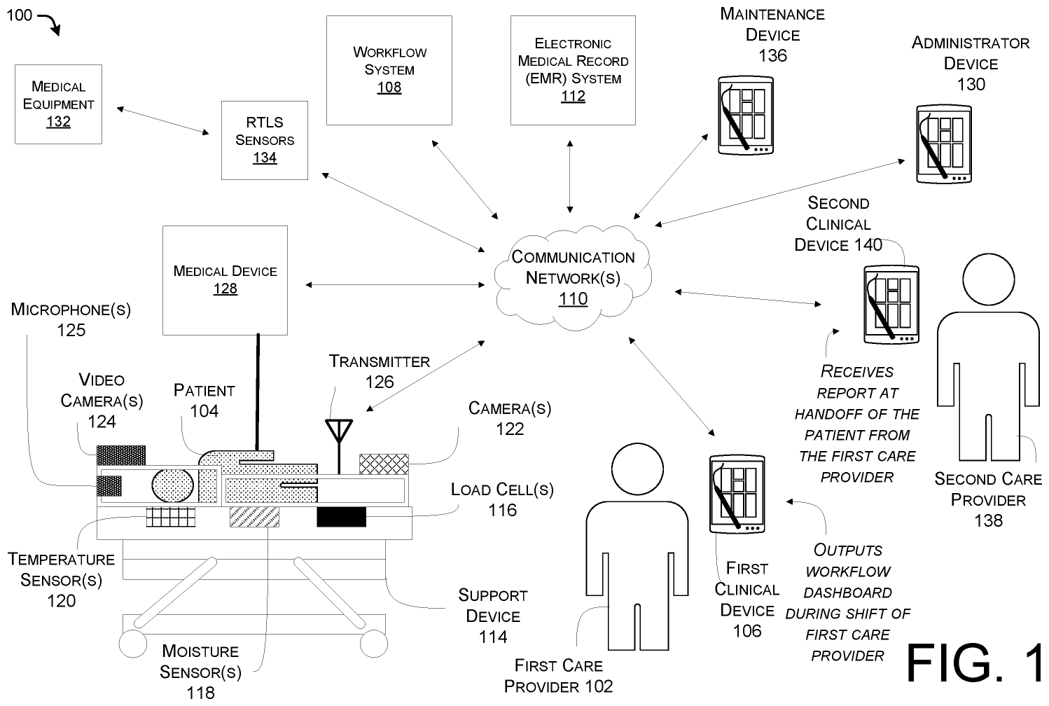

FIG. 1 illustrates an example networking environment 100 for providing assistance to care providers in a clinical environment. The clinical environment, for instance, may be a hospital, a clinic, a care facility, or a combination thereof. A first care provider 102 may be responsible for caring for a patient 104 during a first time period. As used herein, the terms “medical care provider,” “care provider,” and their equivalents, can refer to an individual responsible for monitoring, treating, diagnosing, or managing health care of at least one patient. Examples of care providers include nurses, physicians, physician assistants, therapists (e.g., respiratory therapists, physical therapists, etc.), and medical technicians. As used herein, the term “patient,” and its equivalents, can refer to an individual being monitored and/or cared for within a clinical environment or who has been previously monitored and/or cared for within the clinical environment. In various examples, a patient is a human, but implementations of this disclosure are not so limited. As used herein, the terms “health care,” “medical care,” and their equivalents, can refer to diagnostic and/or therapeutic medical interventions performed on a patient. As used herein, the terms “responsible for,” “assigned to,” and their equivalents, can refer to a relationship between one or more patients and a care provider responsible for monitoring and/or caring for the patient(s). The first time period may be a scheduled shift or workday of the first care provider 102.

The first care provider 102 is assigned, carries, or is otherwise associated with a first clinical device 106. As used herein, the term “clinical device” can refer to a computing device configured to output information to at least one care provider. Examples of clinical devices include mobile phones, tablet computers, personal computers, laptops, smart televisions, or any other computing device configured to output information to a user. In some cases, a clinical device includes a computing device that includes a screen outputting patient status information to multiple care providers, such as a screen listing patient statuses of multiple patients being managed by care providers on a particular floor, wing, or unit within the clinical environment.

A workflow system 108 may be configured to generate a workflow report and to deliver the workflow report to the first clinical device 106 of the first care provider 102. As used herein, the terms “workflow report,” “workflow dashboard,” “dashboard,” and their equivalents refers to a summary of patient statuses and/or tasks associated with patients assigned to a care provider. In some cases, a workflow report represents an ordered list of patients and/or tasks along with information relevant to the care of those patients. For example, although not illustrated in FIG. 1, the first care provider 102 may be responsible for caring for multiple patients including the patient 104, and the workflow report may summarize the patient statuses and/or tasks associated with the multiple patients. The workflow system 108 may be implemented in hardware, software, or a combination thereof. For example, the workflow system 108 may include software being executed on one or more servers located in the clinical environment or remote from the clinical environment.

The workflow system 108 may transmit the workflow report to the first clinical device 106 over one or more communication networks 110. The communication network(s) 110 include wired (e.g., electrical or optical) and/or wireless (e.g., radio access, BLUETOOTH, WI-FI, or near-field communication (NFC)) networks. The communication network(s) 110 may forward data in the form of data packets and/or segments between various endpoints, such as computing devices, medical devices, servers, and other networked devices.

In various implementations, the workflow system 108 may generate and/or update the workflow report based on patient data. As used herein, the term “patient data,” and its equivalents, can refer to digital data indicative of historical and/or current conditions of a patient. In some examples, patient data includes data stored or extracted from an electronic medical record (EMR) of a patient. As used herein, the terms “electronic medical record,” “EMR,” “electronic health record,” and their equivalents, can refer to a data indicating previous or current medical conditions, diagnostic tests, or treatments of a patient. In various cases, one or more servers store EMRs of multiple patients. The EMRs may also be accessible via computing devices operated by care providers. In some cases, data stored in the EMR of a patient is accessible to a user via an application operating on a clinical device. For instance, patient data may indicate demographics of a patient, vital signs of a patient, notes from one or more medical appointments attended by the patient, medications prescribed or administered to the patient, therapies (e.g., surgeries, outpatient procedures, etc.) administered to the patient, results of diagnostic tests performed on the patient, patient identifying information (e.g., a name, birthdate, etc.), or a combination thereof.

The workflow system 108 may receive, from an EMR system 112, data included in an EMR of the patient 104. The workflow system 108 may generate and/or update the workflow report based, at least in part, on the EMR of the patient 104. The EMR system 112 may be implemented in hardware, software, or a combination thereof. For example, the EMR system 112 may include one or more servers hosting at least one database that stores EMRs of multiple patients inside and outside of the clinical environment. The EMR system 112 may transmit the data in the EMR of the patient 104 to the workflow system 108 over the communication network(s) 110. In various examples, the first care provider 102 may access the EMR of the patient 104 on-demand. For instance, the first clinical device 106 may operate an application associated with the EMR system 112, which can receive the data included in the EMR of the patient 104. The first clinical device 106 may also modify or add information to the EMR of the patient 104 stored by the EMR system 112 by transmitting data indicative of the modifications or additions to the EMR system 112.

In some examples, the workflow system 108 may utilize the patient data to monitor key metrics in the environment 100. For example, the workflow system 108 may analyze the patient data in order to monitor results-oriented metrics such as readmissions to the hospital, discharge rates, falls, sepsis, pressure injuries, compliance with particular rules (e.g., Medicare compliance), and the like, within the care facility. In various examples, the workflow system 108 may also analyze the patient data in order to identify whether various tasks associated with patient care were performed, and when they were performed, by care providers in the care facility. The workflow system 108 may generate, based on the results-oriented metrics and the completion of the tasks, a compliance report that is indicative of a compliance of the care providers with respect to various goals of the care facility. The compliance report may be output to an administrator. The administrator, for example, may use the compliance report in order to adjust staffing, number of patients being cared for within the care facility, and/or prioritization of tasks to be performed on the patients, in order to improve patient outcomes.

In some cases, the workflow system 108 may generate the workflow report based on text, audio, and/or video associated with the patient 104, which may or may not be stored in the EMR of the patient 104. For example, the first clinical device 106 may operate a messaging application that receives text, audio, and/or video messages about the patient 104 from the first care provider 102. The first clinical device 106 may transmit the text, audio, and/or video messages to other devices, such as computing devices operated by other care providers, by the patient 104, or by family members of the patient 104. In some cases, the first clinical device 106 may receive text, audio, and/or video messages about the patient 104 from the other computing devices. The messages may include questions and updates about the status of the patient 104.

According to some implementations, the workflow system 108 may generate the workflow report based on data obtained from sensors monitoring the patient 104. The patient data, in some examples, includes data indicating ongoing vital signs or other physiological parameters of the patient 104 as they are monitored. For example, the patient data may include data indicating a vital sign that is streamed or otherwise obtained from a medical device or other type of sensor monitoring the patient 104. As used herein, the term “vital sign,” and its equivalents, can refer to a physiological parameter indicating a medical status of a patient. Vital signs include, for example, temperature (e.g., body temperature), pulse rate, respiration rate, blood pressure, airway CO₂ (e.g., EtCO₂), blood oxygenation (e.g., SpO₂), electrocardiogram (ECG), electroencephalogram (EEG), electrolyte (e.g., sodium and potassium) levels, or a combination thereof.

At least some of the sensors monitoring the patient 104 may be part of or otherwise integrated into a support device 114. The support device 114 includes, for instance, a gurney, hospital bed, or some other structure configured to support the patient 104. As used herein, the terms “bed,” “hospital bed,” and their equivalents, can refer to a padded surface configured to support a patient for an extended period of time (e.g., hours, days, weeks, or some other time period). The patient 104 may be laying down on the support device 114. For example, the patient 104 may be resting on the support device 114 for at least one hour, at least one day, at least one week, or some other time period. In various examples, the patient 104 and the support device 114 may be located in the clinical environment. In some implementations, the support device 114 includes a mechanical component that can change the angle at which the patient 104 is disposed. In some cases, the support device 114 includes padding to distribute the weight of the patient 104 on the support device 114. According to various implementations, the support device 114 can include vital sign monitors configured to output alarms or otherwise communicate vital signs of the patient 104 to external observers (e.g., care providers, family members, and the like). The support device 114 may include railings that prevent the patient 104 from sliding off of a resting surface of the support device 114. The railings may be adjustable, in some cases.

In various examples, the support device 114 includes one or more load cells 116. The load cell(s) 116 may be configured to detect a pressure on the support device 114. In various cases, the load cell(s) 116 can include one or more strain gauges, one or more piezoelectric load cells, a capacitive load cell, an optical load cell, any device configured to output a signal indicative of an amount of pressure applied to the device, or a combination thereof. For example, the load cell(s) 116 may detect a pressure (e.g., weight) of the patient 104 on the support device 114. In some cases, the support device 114 includes multiple load cells that respectively detect different pressures on the support device 114 in different positions along the support device 114. In some instances, the support device 114 includes four load cells arranged at four corners of a resting surface of the support device 114, which respectively measure the pressure of the patient 104 on the support device 114 at the four corners of the support device 114. The resting surface, for instance, can be a surface in which the patient 104 contacts the support device 114, such as a top surface of the support device 114.

The support device 114 may include one or moisture sensors 118. The moisture sensor(s) 118 may be configured to measure a moisture on a surface (e.g., the resting surface) of the support device 104. For example, the moisture sensor(s) 118 can include one or more capacitance sensors, one or more resistance sensors, one or more thermal conduction sensors, or a combination thereof. In some cases, the moisture sensor(s) 118 include one or more fiber sheets configured to propagate moisture to the moisture sensor(s) 118. In some cases, the moisture sensor(s) 118 can detect the presence or absence of moisture (e.g., sweat or other bodily fluids) disposed between the support device 114 and the patient 104.

In various examples, the support device 114 can include one or more temperature sensors 120. The temperature sensor(s) 120 may be configured to detect a temperature of the patient 104 and/or the support structure 114. In some cases, the temperature sensor(s) 120 includes one or more thermistors, one or more thermocouples, one or more resistance thermometers, one or more Peltier sensors, or a combination thereof.

The support device 114 may include one or more cameras 122. The camera(s) 122 may be operably connected to at least one processor had have a field of view. In some examples, the camera(s) 122 may be configured to capture images of the field of view, such as images of the patient 104, the support structure 114, a room in which the patient 104 and support structure 114 are located, or a combination thereof. In various cases, the camera(s) 122 may include radar sensors, infrared cameras, visible light cameras, depth-sensing cameras, or any combination thereof. In some examples, infrared images may indicate, for instance, a temperature profile of the patient 104 and/or the support structure 114. Thus, the camera(s) 122 may be a type of temperature sensor. In addition, the images may indicate a position of the patient 104 and/or the support structure 114, even in low-visible-light conditions. For example, the infrared images may capture a position of the patient 104 during a night environment without ambient lighting in the vicinity of the patient 104 and/or the support structure 114. In some cases, the camera(s) 122 may include one or more infrared video cameras. The camera(s) 122 may include at least one depth-sensing camera configured to generate a volumetric image of the patient 104, the support structure 114, and the ambient environment. According to various implementations, the images and/or videos captured by the camera(s) 122 are indicative of a position and/or a movement of the patient 104 over time.

According to some examples, the support device 114 can include one or more video cameras 124. The video camera(s) 124 may be configured to capture videos of the patient 104, the support structure 114, an entrance to a room containing the support structure 114, an entrance to a bathroom adjacent to the room containing the support structure 114, the room containing the patient 104 and the support structure 114, or a combination thereof. The videos may include multiple images of the patient 104 and/or the support structure 114. Thus, the videos captured by the video camera(s) 124 may be indicative of a position and/or movement of the patient 104 over time. In some examples, the video camera(s) 124 capture visible light videos, changes in radar signals over time, infrared videos, or any combination thereof.

In some examples, the support device 114 can include one or more microphones 125 configured to capture audio signals output by the patient 104, the support device 114, and/or the ambient environment. The audio signals captured by the microphone(s) 125 may be indicative of a position and/or movement of the patient 104 over time. In particular cases, the microphone(s) 125 are integrated within the camera(s) 122 and/or video camera(s) 124.

In some examples, the support structure 114 includes a head rail and a foot rail. The camera(s) 122 and/or video camera(s) 124, for instance, are mounted on the head rail, the foot rail, an extension (e.g., a metal or polymer structure) attached to the head rail or the foot rail, or any combination thereof. In various implementations, the camera(s) 122 and/or video camera(s) 124 are attached to a wall or ceiling of the room containing the support structure 114. In some examples, the camera(s) 122 and/or video camera(s) 124 are attached to a cart or other object that is located in the vicinity of the support structure 114.

In various cases, the sensors (e.g., the load cell(s) 116, the moisture sensor(s) 118, the temperature sensor(s) 120, the camera(s) 122, the video camera(s) 124, or any combination thereof) of the support device 114 are configured to monitor one or more parameters of the patient 104 and to generate sensor data associated with the patient 104. In various cases, the sensors convert analog signals (e.g., pressure, moisture, temperature, light, electric signals, sound waves, or any combination thereof) into digital data that is indicative of one or more parameters of the patient 102. As used herein, the terms “parameter,” “patient parameter,” and their equivalents, can refer to a condition of an individual and/or the surrounding environment. In this disclosure, a parameter of the patient 104 can refer to a position of the patient 104, a movement of the patient 104 over time (e.g., mobilization of the patient 104 on and off of the support device 114), a pressure between the patient 104 and an external object (e.g., the support device 114), a moisture level between the patient 104 and the support device 114, a temperature of the patient 104, a vital sign of the patient 104, a nutrition level of the patient 104, a medication administered and/or prescribed to the patient 104, a previous condition of the patient 104 (e.g., the patient was monitored in an ICU, in dialysis, presented in an emergency department waiting room, etc.), circulation of the patient 104 (e.g., restricted blood flow), a pain level of the patient 104, the presence of implantable or semi-implantable devices (e.g., ports, tubes, catheters, other devices, etc.) in contact with the patient 104, a sound emitted by the patient 104, or any combination thereof. In various examples, the load cell(s) 116, the moisture sensor(s) 118, the temperature sensor(s) 120, the cameras 122, the video camera(s) 124, the microphone(s) 125, or a combination thereof, generates sensor data indicative of one or more parameters of the patient 104.

In various examples, a transmitter 126 is communicatively coupled with the various sensors monitoring the patient 104, such as the load cell(s) 116, the moisture sensor(s) 118, the temperature sensor(s) 120, the camera(s) 122, the video camera(s) 124, and the microphone(s) 125. The transmitter 126 may transmit, to the workflow system 108 over the communication network(s) 110, a signal indicative of the sensor data. In some cases, the transmitter 126 is included in the support device 114. The transmitter 126 may be a transceiver configured to transmit and receive signals from other components in the environment 100 over the communication network(s) 110, in particular examples. In various examples, an artificial intelligence (AI) layer may extract workflow primitives based on the data from the sensors. For example, the AI layer may deduce that the patient 104 has turned by themselves based on the interpretation of the data from the load cell(s) 116, camera(s) 122, and/or video camera(s) 124. A higher level intelligence layer may deduce that a task associated with turning the patient 104 by a care provider (e.g., the first care provider 102) to prevent risk of a pressure injury by the patient 104 is unnecessary or moot.

According to some implementations, a medical device 128 may be generating sensor data by monitoring the patient 104. The medical device 128 may be external to the support device 114, for example. Examples of the medical device 128 include a bedside monitor, such as a blood pressure monitor, an oxygenation monitor, a capnography monitor, a respiratory monitor, a cardiac monitor (e.g., a Holter monitor), or a temperature monitor. The medical device 128 may include an output device (e.g., a screen) that outputs signals indicative of the sensor data captured by the medical device 128. In various implementations, the medical device 128 includes a transmitter configured to transmit the sensor data to the workflow system 108.

In various examples, the workflow system 108 is configured to generate the workflow report based on EMR data from the EMR system 112; message data from one or more clinical devices in the environment (e.g., the first clinical device 106); and sensor data from the support device 114, the medical device 128, and other sensors monitoring the patient 104. In various implementations, the workflow system 108 may generate a summary of a condition of the patient 104 and include the summary in the workflow report. As used herein, the terms “condition,” “status,” “patient status,” and their equivalents, can refer to a state of a patient being cared for in a clinical environment. In some cases, the condition of a patient refers to how the patient is being cared for in the clinical environment, such as the location of the patient, admittance to a ward (e.g., an ICU), the care providers assigned to the patient, and so on. In some cases, the condition of a patient refers to the patient's readiness for a visit from a care provider, such as whether the patient is currently sleeping, undressed, in an appointment with another care provider, and so on. A condition of the patient can refer to information relevant to a medical condition of the patient, such as the reason why the patient is being cared for in the clinical environment, medications, allergies, vital signs, physiological parameters, medical history, procedures performed on the patient, procedures to be performed on the patient, and so on. In some cases, a condition of the patient can refer to a predicted state of a patient. For example, one or more parameters of the patient 104 may indicate that the patient 104 is at risk of developing sepsis within the next eight hours, and the condition of the patient 104 may include the sepsis risk of the patient 104.

In some examples, the workflow system 108 may determine or predict whether the patient 104 is available for a visit from the first care provider 102. The workflow system 108 may determine that the patient 104 is being supported by the support device 114 based on data from the load cell(s) 116 and/or temperature sensor(s) 120 (or in some cases, the camera(s) 122, video camera(s) 124, and/or microphone(s) 125), which may indicate that the patient 104 is in their room within the clinical environment rather than in surgery, in an appointment with a care provider outside of the room, walking around the clinical environment, or using a bathroom, for example. In some cases, other sensors, such as a television (TV) remote sensing a button press by the patient 104, a microphone of a virtual assistant in the vicinity of the patient 104, or the like, can be used to identify that the patient 104 is present within the room. The workflow system 108 may determine that the patient 104 is in the room and/or supported by the support device 114 based on data generated by the camera(s) 122 and/or video camera(s) 124. In some cases, the workflow system 108 may determine that the patient 104 is awake (rather than sleeping) or is in between meal based on the data generated by the camera(s) 122 and/or video camera(s) 124. The workflow system 108 may indicate, in the workflow report, whether the patient 104 is ready for a visit from the first care provider 102.

In some implementations, the workflow system 108 may predict a time at which the patient 104 will be available for a visit from the first care provider 102. For example, the workflow system 108 may determine, based on sensor data acquired over an extended time period (e.g., multiple days), that the patient 104 consistently falls asleep during certain times of the day or eats meals during other times of the day. In some cases, the workflow system 108 may include a predictive model that is configured to predict, based on the sensor time, a time at which the patient 104 will be available. The workflow system 108 may indicate, in the workflow report, a predicted time at which the patient 104 will be available for a visit from the first care provider 102.

According to some cases, the workflow system 108 may indicate, in the workflow report, a risk of the patient 104 in experiencing a complication that could prevent the patient 104 from being discharged from the clinical environment or that could otherwise negatively impact the health of the patient 104. For example, the workflow system 108 may determine, based on the EMR data, the message data, the sensor data, or a combination thereof, a sepsis risk of the patient 104, a fall risk of the patient 104, a probability of the patient 104 being discharged (e.g., a probability of the patient 104 achieving a particular mobility goal, such as unassisted walking), or a pressure injury risk of the patient 104. As used herein, the term “sepsis risk,” and its equivalents, can refer to a likelihood, probability, or susceptibility of a patient to sepsis. Sepsis refers to an overwhelming immune response to an infection. In sepsis, the immune response leads to widespread inflammation that can prevent blood flow from reaching vital organs and can lead to organ damage. In some cases, sepsis causes shock, which can result into the failure of multiple organs and potentially death. The sepsis risk of a patient can be impacted by the patient's immune system and/or susceptibility to infection. Factors impacting sepsis risk include, for instance, invasive procedures performed on the patient, how frequently wound dressings are replaced, invasive devices in contact with the patient, implantable devices implanted in a patient, and so on. The workflow system 108 may identify the sepsis risk of the patient based on the patient data of the patient 104. As used herein, the term “fall risk,” and its equivalents, can refer to a likelihood, probability, or susceptibility of a patient physically falling down and experiencing a serious injury (e.g., broken bone, hemorrhage, etc.). Various factors impact a patient's fall risk, such as whether the patient has a condition (e.g., multiple sclerosis) impacting the patient's balance or steadiness, a condition (e.g., osteoporosis) impacting the patient's susceptibility to serious injury, a condition associated with loss of consciousness (e.g., epilepsy), choking, vomiting, apnea, mental health (e.g., delirium), previous code blues called on the patient, and so on. In some cases, the patient's environment impacts the fall risk, such as whether a bed of the patient has guard rails to prevent a fall, whether the patient is using equipment (e.g., a walker, wheelchair, etc.) preventing falls, friction of a surface (e.g., a floor) supporting the patient, height of the patient, and so on. In various implementations, the workflow system determines the fall risk of the patient 104 based on patient data. In some cases, the workflow system 108 may determine a risk that the patient 104 will experience a pressure injury within a particular time period (e.g., the next day) based on the patient data.

The workflow system 108 may indicate the sepsis risk, fall risk, pressure injury risk, or a combination thereof, of the patient 104 in the workflow report in a variety of ways. For example, the workflow system 108 may output the risk itself as a percentage risk, wherein 0% corresponds to no risk and 100% corresponds to a certainty that the patient 104 will experience the complication (e.g., within a particular time period, such as one day). In some cases, the workflow system 108 may include an icon, color, alarm, or other indicator in the workflow report when the risk is above a particular threshold (e.g., the percentage risk is greater than 50%). According to some implementations, the workflow system 108 may indicate the statuses of multiple patients assigned to the first care provider 102 in the workflow report, and may arrange an order of user interface elements corresponding to the multiple patients in the workflow report based on the respective risks of the patients. For instance, the workflow system 108 may list a patient with a highest risk at the top of a graphical user interface (GUI) of the workflow report and a patient with a lowest risk at the bottom of the GUI.

In some examples, the workflow system 108 generates other status information of the patient 104 based on the patient data and includes the other status information in the workflow report. In some cases, the workflow system 108 determines a bedside mobility assessment tool (BMAT) level of the patient 104 based on the patient data and may include the BMAT level in the workflow report. In some examples, the workflow system 108 determines an early warning score (EWS) of the patient 104 based on the patient data and may include the EWS in the workflow report.

According to various implementations, the workflow system 108 determines one or more tasks associated with the patient 104, which can be completed by the first care provider 102 during the first time period. As used herein, the terms “task,” “clinical task,” and their equivalents refers to an action item to be performed by a care provider in order to manage the care of a patient. A task, for example, can be a diagnostic activity to be performed on the patient (e.g., a vitals sign check, medical imaging, etc.), a therapeutic activity to be performed on the patient (e.g., administration of a medication, physical therapy, a surgery, etc.), an activity to prevent medical complications (e.g., toileting assistance, sheet change, replacement of a port or other invasive and/or semi-invasive device, etc.), and so on.

The workflow system 108 may determine the task(s) based on the patient data. For example, if the sensor data indicates that a vital sign of the patient 104 is outside of a particular range, the workflow system 108 may recommend that the first care provider 102 should check on the patient 104 within a particular time period (e.g., the next half hour) in order to determine whether a therapy (e.g., a medication) should be administered to the patient 104. If the sensor data indicates that the patient 104 has remained in the support device 114 for greater than a threshold time period, the workflow system 108 may recommend that the first care provider 102 visit the patient 104 and encourage the patient 104 to walk throughout the clinical environment and/or that the first care provider 102 assist the patient 104 with toileting. In examples in which the sensor data generated from the moisture sensor(s) 118 indicates that there is moisture between the patient 104 and the support device 114, the workflow system 108 may recommend that the first care provider 102 clean or change sheets on the support device 114.

In some examples, the workflow system 108 includes a computing model that automatically identifies tasks associated with improved patient outcomes based on patient data from the patient 104 and other patients in the clinical environment. As used herein, the term “computing model,” and its equivalents, refers to a computational model that accepts input data and that provides output data based on the input data. In some examples, a computing device, when executing the computing model, will analyze the input data based on the computing model and the input data. In some cases, a computing model includes at least one mathematical model or function.

The computing model, for instance is a machine learning model. As used herein, the term “machine learning model,” and its equivalents, can refer to a type of computing model that is built and/or optimized based on training data. The process of building or optimizing a machine learning model is referred to as “training.” Once a machine learning model is trained, a computing device executing the machine learning model is configured to generate output data based on new input data, which may be different than the training data. The computing model in the workflow system 108, for example, may be trained using patient data obtained from previous patients of the clinical environment as well as the health outcomes (e.g., mortality, morbidity, discharge schedule, complication records, etc.) of those previous patients. The computing model may identify tasks completed by care providers correlated with improved outcomes (e.g., lower mortality, lower morbidity, quicker time to discharge, fewer complications, etc.) of the previous patients. The computing model may then accept the patient data of the patient 104 as input and determine what tasks can be completed on the patient 104 that may be associated with improved outcomes for the patient 104.

In some cases, the workflow system 108 may determine the task(s) associated with the patient 104 based on one or more predefined rules associated with the clinical environment. For example, an administrator device 130 (e.g., controlled by a user associated with the clinical environment) may transmit one or more task rules to the workflow system 108. In some cases, each task rule may specify an if-then statement indicating that a task should be performed on a patient if one or more events are observed. For example, a task rule may indicate that each patient should be encouraged to walk within four hours after having a particular surgery (e.g., appendectomy). Accordingly, organizations can customize the types of tasks to assign care providers in the clinical environment based on public health knowledge, for instance.

The workflow system 108 may further determine whether medical equipment 132 is associated with the task(s). The medical equipment 132, for example, is any object that can be used to diagnose, treat, or manage the patient 104. For instance, the medical equipment 132 may be mobility equipment (e.g., a wheelchair, gurney, walker, etc.), a therapeutic medical device (e.g., a continuous positive airway pressure (CPAP) machine), a diagnostic medical device (e.g., a portable ultrasound machine, a thermometer, etc.), and so on. If the workflow system 108 determines that a particular task can be performed with the medical equipment 132, the workflow system 108 may determine whether the medical equipment 132 is available and/or located within a vicinity of the patient 104. In some cases, the medical equipment 132 includes a real time location system (RTLS) tag that emits a wireless signal (e.g., an NFC signal). The wireless signal may be received by RTLS sensors 134 located at predetermined locations in the clinical environment. The RTLS sensors 134 may transmit, to the workflow system 108, one or more signals indicative of the times at which the wireless signal from the medical equipment 132 is received. The workflow system 108 may determine the location of the medical equipment 132 based on the signal(s) from the RTLS sensors 134. The workflow system 108 may also determine the location of the patient 104, e.g., based on the patient data. In some cases, the workflow system 108 may determine whether the medical equipment 132 is located within a threshold distance of the location of the patient 104 or within a room associated with the patient 104. The workflow system 108 may indicate, in the workflow report, whether the medical equipment 132 is in the vicinity of the patient 104.

In some cases, the workflow system 108 may facilitate movement of the medical equipment 132 to the vicinity of the patient 104. For example, the workflow system 108 may indicate the location of the medical equipment 132 in the workflow report, such that the first care provider 102 may efficiently acquire the medical equipment 132 at the location prior to visiting the patient 104 to complete the associated task.

The workflow report may be output by the first clinical device 106 in any of a variety of ways. For example, the workflow report may be output as a GUI on a screen of the first clinical device 106. The workflow report may at least partially output as audio signals from a speaker of the first clinical device 106 or as vibration from a haptic element of the first clinical device 106. According to some examples, the workflow report may be output via an application operating on the first clinical device 106. In some cases, the workflow report is output as a plug-in application of an EMR application through which the first care provider 102 can access the EMR of the patient 104.

In some cases, the workflow report may be displayed by the first clinical device 106 in different views. In a patient view, the workflow report may include graphical elements respectively associated with the patients (including, e.g., the patient 104) assigned to the first care provider 102. For instance, the patient view may include a list of the patients assigned to the first care provider 104 as well as their respective statuses and tasks due. The workflow report may also be output in a task view. In the task view, the workflow report may include graphical elements respectively associated the tasks associated with the patients of the first care provider 102. For example, the workflow report may include a graphical list of tasks associated with the patient 104 to be completed by the first care provider 102 during the first time period. In some cases, the graphical elements in the patient view and/or the graphical elements in the task view are visually arranged by priority. In some examples, the graphical elements associated with priority patients and/or tasks are displayed differently than other graphical elements. For instance, the graphical elements may illustrate the priority based on color, blinking, and so on. In various implementations, the workflow system 108 may generate the workflow report to summarize tasks for the first care provider 102, and arrange those tasks according to a priority, based on a set of pre-set or actively or passively learned set of rules.

In various implementations, the first clinical device 106 may receive input signals from the first care provider 102 related to the patient 104 and/or tasks. For example, the workflow report may be output on a touchscreen of the first clinical device 106, which may display a graphical element associated with a particular task (e.g., “check vital signs”) associated with the patient 104. The first care provider 102 may indicate that the particular task has been completed by touching a user interface element (e.g., a button) displayed on the touchscreen. The first clinical device 106, in various examples, may transmit a signal to the workflow system 108 indicating that the task has been completed. The workflow system 108 may update the workflow report based on the signal. For example, the workflow system 108 may remove a graphical element associated with the completed task from the workflow report.

The workflow system 108 may update the workflow report in real-time based on updated patient data and/or input from the first clinical device 106. If the patient data indicates that a status of the patient 104 has changed, the workflow system 108 may indicate the changed status in the workflow report. In addition, the workflow report 108 may be updated based on changes in the location of the medical equipment 132.

In some cases, the workflow report includes a user interface element that enables the first care provider 102 to order the medical equipment 132 to be brought to the vicinity of the patient 104. For example, the workflow system 108 may initially determine that the medical equipment 132 is located outside of the vicinity of the patient 104. The first care provider 102 may select an option to order the medical equipment 132. In response, the workflow system may transmit a request to relocate the medical equipment 132 to the vicinity of the patient 104 (e.g., to the room of the patient 104) to a maintenance device 136, which may be associated with a non-care provider user. The user, upon receiving the request at the maintenance device 136, may physically move the medical equipment 132 to its desired location. The workflow system 108 may detect that the medical equipment 132 has been relocated to a vicinity of the patient 104 and may inform the first care provider 102 via the workflow report (e.g., as a pop-up notification). Thus, the first care provider 102 may not have to take time out of their workday or shift to relocate the medical equipment 132. In some cases, the first care provider 102 may further indicate, through the workflow report, that the medical equipment 132 requires cleaning or maintenance after use. The workflow system 108 may, in response to receiving the indication, provide a request to the maintenance device 136 requesting the cleaning or maintenance.

In some cases, the first care provider 102 may update the EMR of the patient 104 using the workflow report. For example, the first care provider 104 may enter a note associated with the patient 104 in the workflow report, which may be forwarded to the EMR of the patient 104 by the workflow system 108. Further, the workflow report may be updated based on data from the EMR. Thus, the EMR and the workflow report may be seamlessly integrated.

In various implementations of the present disclosure, the workflow system 108 may further facilitate handoff from the first care provider 102 to a second care provider 138. As used herein, the terms “handoff,” “handover,” and their equivalents, can refer to a transition of care of one or more patients from one care provider to another care provider. Handoff, for example, occurs during a shift change, during transfer of a patient between facilities, during ward changes, and other events in which care for the patient is transferred between care providers. In particular examples, the workflow system 108 may act as an information broker system between the first care provider 102 and the second care provider 138, to ensure that the second care provider 138 has enough information about the patient 104 to avoid interruptions in care at handoff.

The workflow system 108 may generate a handoff report based on the patient data. As used herein, the terms “handover report,” “handoff report,” “report,” “message,” and their equivalents refers to a collection of data, configured to be output to a user, that summarizes medically relevant information about one or more patients whose care is being transferred from one care provider to another care provider. The workflow system 108, for example, generates the handoff report based on the sensor data (e.g., obtained from and/or captured by at least one of the load cell(s) 116, the moisture sensor(s) 118, the temperature sensor(s) 120, the camera(s) 122, the video camera(s) 124, the microphone(s) 125, or the medical device 128), the EMR data form the EMR system 112, and message data to and from clinical devices (e.g., the first clinical device 106) in the environment 100.

In various cases, the workflow system 108 includes a computing model configured to extract a pertinent subset of the patient data and generate the handoff report based on the pertinent subset. The computing model, for example, is a machine learning model. In some examples, the workflow system 108 trains the computing model to extract the pertinent subset of the patient data based on training data that includes patient data from other patients, as well as various health outcomes associated with those patients. The computing model may be trained to identify features in the patient data associated with particular health outcomes. For example, the computing model may identify types of patient data correlated with falls, sepsis, and other negative health outcomes. In some cases, the computing model may identify types of patient data correlated with positive health outcomes, such as below-average times to discharge. Once trained, the computing model may extract the pertinent subset of the patient data of the patient 104 based on the types of patient data correlated with the negative and positive health outcomes.

The workflow system 108 may generate the handoff report to summarize the status of the patient 104 based on the pertinent subset of the data. In various cases, the handoff report summarizes elements of the status of the patient 104 that are relevant to a second time period corresponding to the workday or shift of the second care provider 138. The handoff report may include various components. As used herein, the terms “report component,” “report portion,” “report section,” and their equivalents, can refer to an element of a handover report. A handover report, for example, includes one or more report components. Examples of report components include patient identifying information (e.g., name, identifier, bed identifier, room identifier, etc.), lists (e.g., summarizing tasks associated with the care of one or more patients to be completed during a shift), timelines (e.g., summarizing recommended deadlines or orders of tasks to be completed during a shift), care information (e.g., identifying one or more care providers providing care to one or more patients, scheduled procedures to be performed on individual patients), and other information relevant to the care of patients in a clinical setting.

In some examples, the handoff report identifies the sickest patient (e.g., the patient with the worst condition) among multiple patients that is being transferred to the second care provider 138 (In some examples, the handoff report indicates a guideline, that indicates why the patient 104 is in the care facility and at least one condition relevant to the care of the patient 104. According to some examples, the handoff report may indicate active issues associated with the patient 104, including what has happened to the patient 104 during the shift of the first care provider 102. In some examples, the handoff report indicates if-then contingency planning, such as an instruction to perform a particular therapy and/or diagnostic test if a condition is observed during the shift of the second care provider 138. In some cases, the workflow system 108 automatically provides prompts to the first care provider 102 for information that the workflow system 108 deems pertinent to the handoff report. According to some implementations, the workflow system 108 acquires patient data continuously and/or periodically throughout the shift of the first care provider 102, generate and/or update the handoff report during the shift of the first care provider 102 based on the patient data, review the patient data, validate the patient data (e.g., identify anomalies in the patient data and prompt the first care provider 102 to validate anomalous patient data), and so on, such that the handoff report is complete and ready to be provided to the second care provider 138 at the end of the shift of the first care provider 102. In some examples, the workflow system 108 receives data indicative of a verbal handoff from the first care provider 102 to the second care provider 138 (e.g., from the first clinical device 106) and may automatically prompt the first care provider 102 (e.g., by transmitting signals to the first clinical device 106 instructing the first clinical device 106 to output the prompt) to clarify details omitted in the verbal handoff.

The workflow system 108 may determine one or more report components that are relevant to the second care provider 138 and may generate the handoff report to include the report component(s). The workflow system 108 may provide the handoff report to a second clinical device 140 associated with the second care provider 138. In particular examples, the workflow system 108 may prompt the first care provider 102 to validate the handoff report before it is provided to the second care provider 138. For example, the first care provider 102 may validate the handoff report by pressing a button of the first clinical device 106, providing a verbal validation that is input into the first clinical device 106 (e.g., a microphone of the first clinical device 106), through an instant messaging service connected to the workflow system 108, via a mobile application operating on the first clinical device 106, or the like. In some cases, the workflow system 108 provides an unvalidated copy of the handoff report to the second care provider 138 in a draft form. According to some cases, the workflow system 108 provides a copy of the handoff report to one or more family members of the patient 104, who can validate the contents of the handoff report before it is provided to the second care provider 138. In some examples, the workflow system 108 stores multiple, previous handoff reports involving the patient 104 that are provided to the first clinical device 106 and the first care provider 102 on demand.

In various examples, the workflow system 108 may facilitate order transfers as the care of the patient 104 is transferred from the first care provider 102 in a first care facility and/or ward to the second care provider 138 in a second care facility and/or ward. The workflow system 108 may store indications of various capabilities (e.g., medications available, equipment available, etc.) at various care facilities and/or wards. The workflow system 108 may translate an order associated with the patient 104 based on the capabilities of the first care facility and/or ward to the capabilities of the second care facility and/or ward. For example, if the patient 104 has an order associated with ongoing therapy from ventilator in the first care facility and/or ward, the workflow system 108 may automatically order a ventilator to be provided to the patient 104 in the second care facility and/or ward. In some cases, the ventilators in the different care facilities and/or wards may have different models and/or manufacturers, but the workflow system 108 may identify a ventilator in the second care facility and/or ward with a capability that matches the order for the patient 104. Thus, the workflow system 108 may map orders for patients transferred between different care facilities and/or wards based on the capabilities in the care facilities and/or wards. In some cases, the workflow system 108 is able to identify mistakes in an order manually transferred from one care facility and/or ward to another and inform the recipient care facility and/or ward of a true status of the patient 104 in order to prevent mistakes with transferring the patient 104 between care facilities and/or wards. These and other features may be indicated in the handoff report to the second care provider 138.

The second care provider 138 may interact with the handoff report via the second clinical device 140. For example, the handoff report may visually output by a touchscreen of the second clinical device. In some cases, the handoff report may be output as a plug-in of the EMR application on the clinical device 140. The second clinical device 140 may receive input signals from the second care provider 138 in response to the handoff report. For example, the second care provider 138 may touch a graphical element in the handoff report that is output by the touchscreen of the second clinical device 140 to obtain additional details about the patient 104. In some cases, the second clinical device 140 may retrieve additional patient data (e.g., EMR data, message data, or sensor data) based on receiving an input signal from the second care provider 138.

In some implementations, the handoff report may act as a “personal digital assistant” to help bring the second care provider 138 up-to-speed on the patients. In examples in which the second care provider 138 would like additional patient data about patient 104, the second care provider 138 may input an inquiry (e.g., verbal or written) into the second clinical device 140. The second clinical device 140 may forward the inquiry to the workflow system 108, which may analyze the inquiry and generate a response based on the inquiry. In some cases, the workflow system 108 may perform natural language processing on the inquiry in order to generate the response. The workflow system 108 may provide the response via the handoff report. For example, the second care provider 138 may verbally input “Is there a chance that patient 104 is at risk of sepsis?” into a microphone of the second clinical device 140, which may output a signal indicative of the verbal input to the workflow system 108. The workflow system 108 may include a computing model configured to identify words, such as “chance,” “patient 104,” and “sepsis,” and may also identify the meaning of the inquiry. In response, the workflow system 108 may evaluate previously obtained patient data of the patient 104 relevant to the sepsis risk of the patient. The workflow system 108 may determine that there is a 60% risk of the patient 104 developing sepsis within the second time period. The workflow system 108 may output, via the handoff report, an indication of the 60% risk. In some cases, the workflow system 108 may also recommend a task associated with the sepsis risk, such as a recommendation to administer prophylactic antibiotics to the patient 104 to reduce the sepsis risk. In various implementations, the workflow system 108 may work like a virtual assistant in answering questions of the second care provider 138 after the first care provider 102 has left the premises. The workflow system 108 may, when prompted by the second care provider 138, share various details about the condition of the patient 104 in a manner similar to a conversation.

According to some implementations, the second care provider 138 may pre-select the report component(s) of the handoff report via entering an input signal into the second clinical device 140. The second clinical device 140 may transmit a signal indicative of the input signal to the workflow system 108, which may be used to generate the handoff report. Thus, if the second care provider 138 would prefer that greater or fewer report components were included in the handoff report, the workflow system 108 can accommodate that request.

In some examples, the workflow system 108 includes another computing model configured to identify what information, in the handoff report, is specifically pertinent to the second care provider 138. The information, for instance, includes one or more report components. The computing model is a machine learning model in some cases. The computing model may be trained to take in training data indicating previous interactions between the second clinical device 140 and the second care provider 138, such as input signals received by the second clinical device 140 and output signals provided by the second clinical device 140. The input signals may be indicative of what sort of information the second care provider 138 has found relevant during previous workdays and shifts. In some cases, the second clinical device 140 may determine if the second care provider 138 accesses the EMR of the patient 104 in response to viewing the handoff report. In particular examples, the second clinical device 140 may determine that the second care provider 138 consistently looks up EMR data (e.g., previous vital signs) of previous patients that are not included in the handoff report. The workflow system 108 may adjust the components within the handoff report accordingly. For example, the workflow system 108 may add the additional EMR data to the handoff report, saving the second care provider 138 the time in looking up the EMR data of the patient 104. In some cases, the workflow system 108 generates and/or adjusts the handoff report based on non-EMR data, such as sensor data and/or message data that is not stored in the EMR system 112. According to some examples, the computing model may generate a template associated with the second care provider 138 that indicates relevant components of the handoff report for the second care provider 138. According to some examples, the template is stored in a database and can be accessed in advance of generating a new handoff report for the second care provider 138.

In some examples, the handoff report is used to track patients during ward change events. For example, if the patient 104 or the second care provider 138 is transferred from one ward to another, the handoff report may provide the second care provider 138 with enough pertinent information about the patient 104 to prevent interruptions in care. In particular examples, the handoff report prompts the second care provider 138 to distribute an existing medication regimen to the patient 104. In various examples, the handoff report may inform the second care provider 138 about workflow, medical device, and medication changes associated with the patient 104.

Although FIG. 1 is described with reference to a single patient 104, in various implementations, the environment 100 includes multiple patients, such as five patients, ten patients, 50 patients, 100 patients, and so on. Using similar techniques to those described above, the workflow system 108 can generate and/or update a workflow report or a handoff report that summarizes the conditions of multiple patients in the environment 100. In some examples, more than two clinical devices are included in the environment 100. Thus, the workflow system 108 can generate multiple workflow reports and/or handoff reports and may provide the reports to various clinical devices within the environment 100.

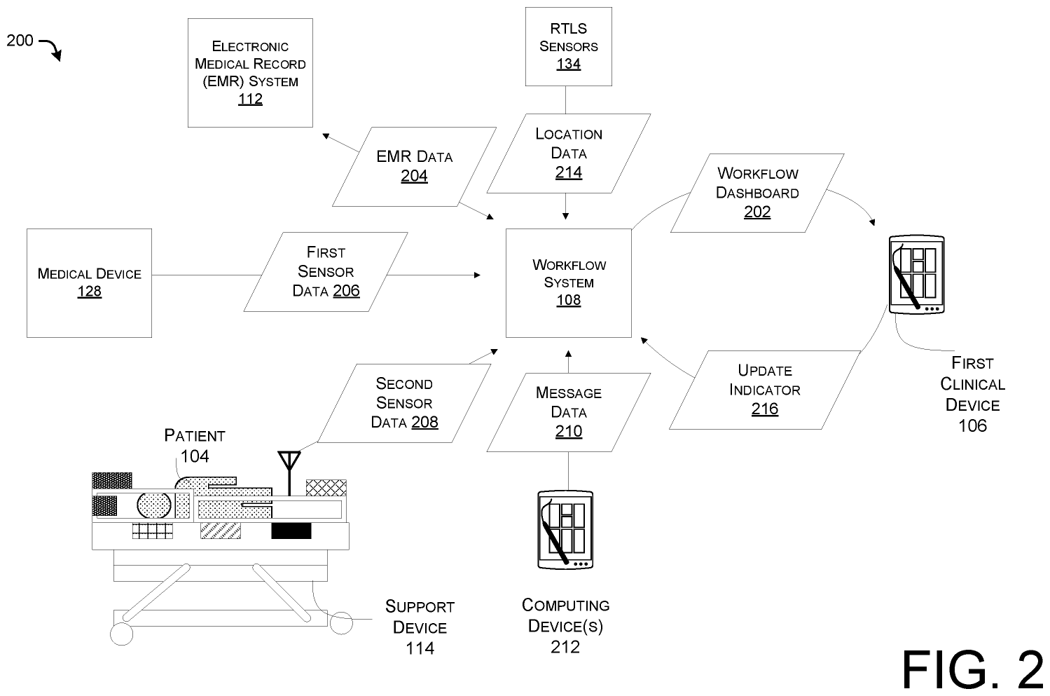

FIG. 2 illustrates example signaling 200 for providing a workflow report 202 to the first clinical device 106. As shown, the signaling 200 is between the first clinical device 106, the workflow system 108, the EMR system 112, the support device 114, the medical device 128, and the RTLS sensors 134 described above with respect to FIG. 1.

The workflow system 108 may generate the workflow report 202 based on patient data, which may be obtained from a variety of sources. For example, the workflow system 108 may generate the workflow report 202 based, at least in part, on EMR data 204 from the EMR system 112. The EMR data 204 may include data extracted from the EMR of the patient 104, such as data indicating one or more parameters of the patient 104, a medical history of the patient 104, demographics of the patient 104, and so on.

The workflow system 108 may generate the workflow report 202 based, at least in part, on first sensor data 206 and second sensor data 208. The first sensor data 206 may be obtained from the medical device 128 monitoring the patient 104. The second sensor data 208 may be obtained from one or more sensors integrated into the support device 114 of the patient 104. Thus, the first sensor data 206 and/or second sensor data 208 may be indicative of a real-time condition of the patient 104, such as a parameter of the patient 104 sampled at a particular frequency (e.g., every 30 seconds, every minute, etc.). In some cases, the first sensor data 206 and the second sensor data 208 are not stored in the EMR system 112.

The workflow system 108 may generate the workflow report 202 based, at least in part, on message data 210 received from one or more computing devices 212. In some examples, the computing device(s) 212 include the first clinical device 106. The message data 210 includes messages related to the patient 104. For example, the message data 210 may include text messages, e-mails, images, videos, audio files, and other types of messages, related to the patient 104. In some cases, the message data 210 may include messages transmitted between multiple computing devices 212 associated with care providers, family members of the patient 104, other people managing care or medical decision-making of the patient 104, the patient 104 themselves, or a combination thereof. In some cases, the message data 210 is not stored in the EMR system 112.

The workflow system 108 may further generate the workflow report 202 based, at least in part, on location data 214 received from the RTLS sensors 134. The location data 214 may indicate the location of equipment within a clinical environment. The workflow system 108 may determine whether the location of the equipment is located within the vicinity of the patient 104 (e.g., within a threshold distance of the patient 104, within a room of the patient 104, etc.). In various cases, the workflow system 108 may indicate whether the equipment is located within the vicinity of the patient 104 in the workflow report 202.

In various examples, the workflow system 108 may generate the workflow report 202 based on one or more care guidelines, which may be stored at the workflow system 108. Care guidelines, for example, can include standard operating procedures and/or procedures set by an administrator of the clinical environment (e.g., a hospital administrator). The workflow system 108 may utilize the care guideline(s) to identify and/or prioritize tasks within the workflow report 202.

The workflow system 108 may provide the workflow report 202 to the first clinical device 106. The workflow report 202 may indicate a status of the patient 104 as well as one or more clinical tasks to be performed for the patient 104. In some cases, the workflow report 202 may indicate statuses of multiple patients (including the patient 104) and tasks associated with the multiple patients. The clinical device 106 may output the workflow report 202 to a user, such as a care provider caring for the patient 104. In addition, the clinical device 106 may update the workflow report 202 based on ongoing patient data associated with the patient 104 and received by the workflow system 108.

Further, the first clinical device 106 may transmit an update indicator 216 to the workflow system 108, which the workflow system 108 may use to update the workflow report 202. The first clinical device 106, for example, may generate the update indicator 216 based on an input signal received from the user. In some cases, the update indicator 216 may indicate a change in the status of the patient 104 or a completed task associated with the patient 104. Based on the update indicator 216, the workflow system 108 may update the workflow report 202 to indicate that the change in the status or that the task has been completed. In some cases, the workflow system 108 may further transmit an indication of the changed status and/or the completed task to the EMR system 112, which may update the EMR of the patient 104 based on the changed status and/or completed task. Thus, the EMR of the patient 104 can be updated based on the workflow report 202.

In particular examples, the workflow system 108 may ensure that the workflow report 202 is up-to-date, even if the care provider associated with the first clinical device 106 has taken a break from caring for the patient 104 or is otherwise interrupted from working with the patient 104. For instance, a care provider may not remember the context that they left the patient 104. In various examples, the workflow system 106 can generate and/or update the workflow dashboard 202 to be used to help the care provider remember an interrupted workflow (e.g., task or multiple tasks) associated with the patient 104, prioritize tasks for the patient 104, and resume them.

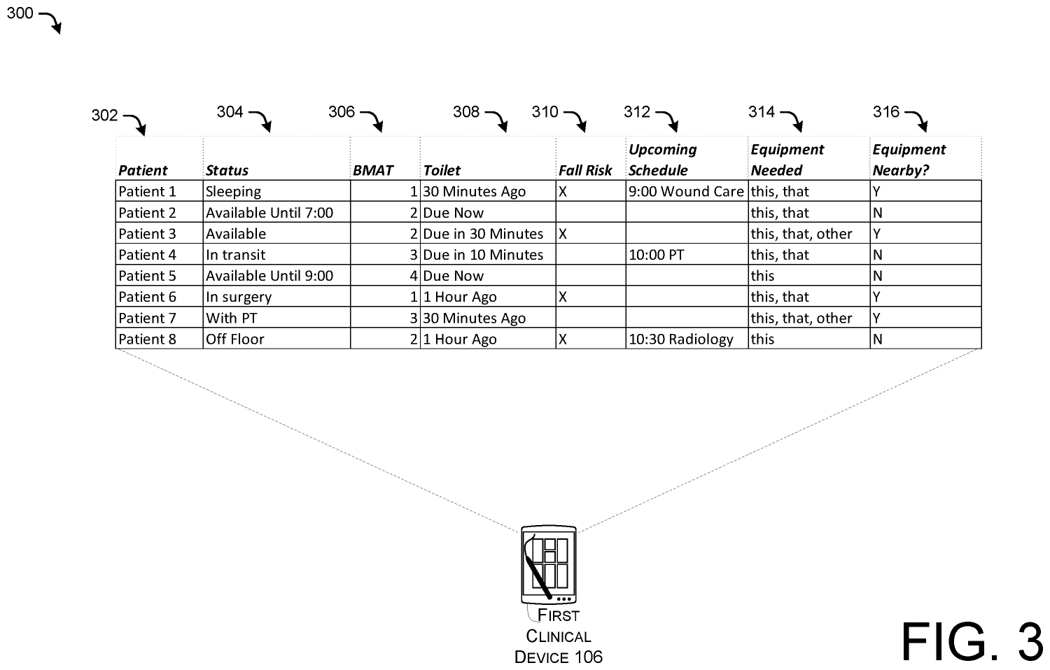

FIG. 3 illustrates an example GUI 300 of the workflow report in a patient mode. As shown, the GUI 300 can be represented as a table ordered by patients assigned to a care provider. The table includes a number of data fields, such as a patient 302 field, a status 304 field, a BMAT 306 field, a toilet 308 field, a fall risk 310 field, an upcoming schedule 312 field, an equipment needed 314 field, and an equipment nearby 316 field. As indicated in the column of the GUI 303 representing the patient 302 field, Patient 1 through Patient 8 are assigned to the care provider. The patient 302 field may indicate an identifier of Patient 1 through Patient 8, such as names, locations, ID numbers, or other identifying information.

The status 304 field indicates an up-to-date condition of each patient. In the example of FIG. 3, the status 304 field indicates an availability status of each patient, but implementations are not so limited. For instance, the status 304 field could indicate a medical status (e.g., one or more vital signs or other physiological parameters) of each patient. The GUI 300 indicates that Patient 1 is currently “Sleeping,” and is therefore unable to receive a visit from the care provider. Patient 3, however is currently “Available” and is ready to receive a visit from the care provider.

The BMAT 306 field indicates a BMAT level of each of the patients. The BMAT level represents an ability of a patient to move safely. The BMAT of a patient, for example, is stored in an EMR of the patient. In various examples, the BMAT level is based on a mobility assessment performed on the patient. At BMAT level 1, the patient has demonstrated the ability to sit and to shake an upper extremity. At BMAT level 2, the patient has demonstrated the ability to stretch (evidencing potential quad muscle strength to stand) and point (evidencing foot drop). At BMAT level 3, the patient has demonstrated the ability to stand and maintain balance for at least five seconds without assistance. At BMAT level 4, the patient has demonstrated the ability walk, advance, and return step. Thus, a care provider may refer to the BMAT 306 field in order to determine a level of mobility assistance to provide to each of the patients. For example, the GUI 300 indicates that Patient 2 has a BMAT level of 2 and Patient 5 has a BMAT level of 4, such that a care provider referring to the GUI 300 can infer that Patient 2 may need a bedpan for toileting and Patient 5 may use minimal assistance for toileting.

The toilet 308 field indicates an upcoming toileting task associated with each patient. A toileting task, for example, may be to assist a patient with elimination. In some cases, a toileting task may be to assist the patient to walk to a toilet, to provide or replace a bedpan of the patient, to check or place a urinary catheter of the patient, to replace a bag attached to a urinary catheter of the patient, to change a diaper of the patient, to check an incontinence pad of the patient, to change an incontinence pad of the patient, to check or change briefs of the patient, or to check a urinary output of the patient. For instance, the GUI 300 may indicate to a care provider that Patient 5 could use immediate toileting assistance and that Patient 4 was provided toileting assistance 30 minutes ago.

The fall risk 310 field indicates a likelihood that each patient will experience significant morbidity by falling down. The fall risk of a patient depends on a likelihood that the patient will fall down. For instance, the a patient's risk of falling down can be dependent on the patient's vision, balance (e.g., diagnosis of an inner ear condition), neurological state (e.g., diagnosis of a neurological condition, like multiple sclerosis, Parkinson's disease, etc.), blood pressure, blood sugar level, electrolyte level, gait, medications, and so on. The fall risk of the patient further depends on a likelihood that the fall will result in a serious injury, such as a broken bone. For example, the patient may be likely to experience a serious injury based on a weight of the patient, a bone density of the patient (e.g., whether the patient has been diagnosed with osteoporosis), a height from which the patient falls, and others. In some cases, the fall risk 310 of each patient is calculated via a computing model or manually by a care provider. In some examples, the fall risk 310 field indicates whether each patient has greater than a threshold fall risk. For example, the GUI 300 may indicate that Patient 3 has a relatively high fall risk and guard rails should be secured on a support structure (e.g., hospital bed) of Patient 3.

The upcoming schedule 312 field indicates scheduled events associated with the patient. For example, a patient may have an appointment scheduled with a specialist care provider within a clinical environment. A care provider may refer to the upcoming schedule 312 field to avoid visiting the patient during an appointment with another care provider. In some cases, the care provider may refer to the upcoming schedule 312 field to assist the patient with transport to the appointment. For example, a care provider referring to the GUI 300 may refrain from attending to the upcoming toileting task for Patient 4 during the 10:00 appointment with a physical therapist (PT).

The equipment needed 314 field indicates physical tools for completion of tasks associated with the patients. For example, a scheduled task may involve the use of mobility equipment to assist with moving a patient, the use of a medical device to diagnose or treat the patient, or a tool (e.g., a physical therapy tool) to perform therapy on the patient. As shown in FIG. 3, a care provider may use “this” to perform a task on Patient 5 and may use “this, that, other” to perform one or more tasks on Patient 7.

The equipment nearby 316 field indicates whether the physical tools for completion of the tasks are located within the vicinities of the patients. In some examples, the equipment nearby 316 field indicates whether equipment is located in the same room as a corresponding patient or whether the equipment is located within a threshold distance of the patient. Thus, the care provider may refer to the GUI 300 to efficiently identify that “this” should be acquired before visiting Patient 5, but “this, that, other” are already located in the vicinity of Patient 7.

In various implementations, the GUI 300 is customizable by the care provider or an administrator. For example, the care provider may determine that the BMAT 306 is unhelpful and remove the BMAT 306 field from the GUI 300. In some cases, the care provider may include other fields associated with monitoring the patient. For example, the care provider may be concerned that Patient 1 to Patient 8 are at risk for a particular complication (e.g., sepsis) that is evidenced by fever, and may add a field within the GUI 300 indicating body temperatures of Patient 1 to Patient 8.

Although not illustrated in FIG. 3, in some examples, the GUI 300 can include other elements that facilitate the workflow of a care provider. For example, the GUI 300 may include a field indicative of times and/or schedules for turning (or otherwise moving) Patient 1 to Patient 8 in order to prevent Patient 1 to Patient 8 from developing pressure injuries. In some cases, sensor data (e.g., data generated from load cells) in the beds of Patient 1 to Patient 8 may be used to identify whether any of the patients have self-turned without assistance and/or have been turned by other care providers. The field in the GUI 300 indicative of times and/or schedules for turning the patients may be updated based on the sensor data.

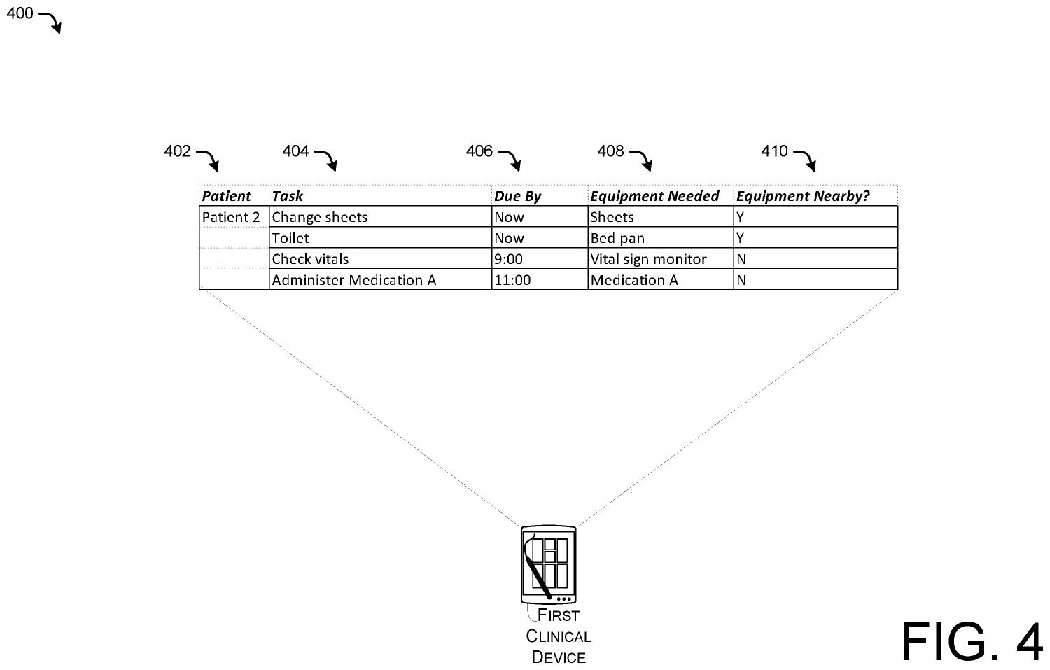

FIG. 4 illustrates an example GUI 400 of the workflow report in a task mode. As shown, the GUI 400 can be represented as a table ordered by tasks to be performed on a patient (e.g., Patient 2) assigned to a care provider. The table includes a number of data fields, such as a patient 402 field, a task 404 field, a due by 406 field, an equipment needed 408 field, and an equipment nearby 410 field. The patient 402 field may indicate an identifier of Patient 2, such as a name, location, ID number, or other identifying information of Patient 2.

The task 404 field may indicate tasks associated with Patient 2 that are to be completed by the care provider. For example, in this example, the care provider is responsible for changing sheets, toileting, checking vitals, and administering a medication (Medication A) to Patient 2. In some cases, the GUI 400 can indicate various tasks, such as a diagnostic activity to be performed on the patient (e.g., a vitals sign check, medical imaging, etc.), a therapeutic activity to be performed on the patient (e.g., administration of a medication, physical therapy, a surgery, etc.), an activity to prevent medical complications (e.g., toileting assistance, sheet change, replacement of a port or other invasive and/or semi-invasive device, etc.), and so on.

The due by 406 field may indicate anticipated or recommended timing of the tasks associated with Patient 2. For example, the workflow report may recommend that the care provider change the sheets and toilet Patient 2 immediately, check vitals at 9:00, and administer Medication A at 11:00. In various cases, the tasks shown in the GUI 400 are ordered based on the due by 406 field, in order to provide a prioritized listing of the tasks to the care provider.

The equipment needed 408 field may indicate what equipment is associated with the pending tasks. For example, the care provider may change the sheets of Patient 2 using “sheets,” the care provider may toilet Patient 2 using a “bed pan,” the care provider may check the vital signs of Patient 2 using a “vital sign monitor,” and my administer the medication using “Medication A.”

The equipment nearby 410 field may indicate whether the equipment is located in the vicinity (e.g., the room or within a threshold distance) of Patient 2. For instance, the sheets and bed pan may be located nearby Patient 2. However, the GUI 400 may recommend that the care provider obtain the vital sign monitor and Medication A prior to traveling to the bedside of Patient 2 in order to complete the “check vitals” and “administer Medication A” tasks.

In some cases, graphical elements associated with the tasks within the GUI 400 are selectable, such that the care provider can indicate, to the workflow system, that a task is completed by selecting the associated graphical element. For instance, a graphical button is output on a touchscreen of a clinical device and is overlapping or adjacent to a row of the GUI 400 corresponding to a particular task. A care provider can indicate that the task is complete by touching the associated graphical button.

In some examples, the GUI 400 presents tasks in a customized order. For example, a champion care facility (e.g., a hospital that is well-respected in its field) may demonstrate that prioritizing one type of task (e.g., turning a patient) over another type of task (e.g., checking vitals) may provide better overall health outcomes for patients cared for by that care facility. In various examples, the workflow system 108 may receive indications that prioritizing the first type of task over the second type of task. In some cases, the tasks of the GUI 400 are arranged based on that prioritization. Thus, the improved results of the champion care facility can be automatically implemented at other care facilities.

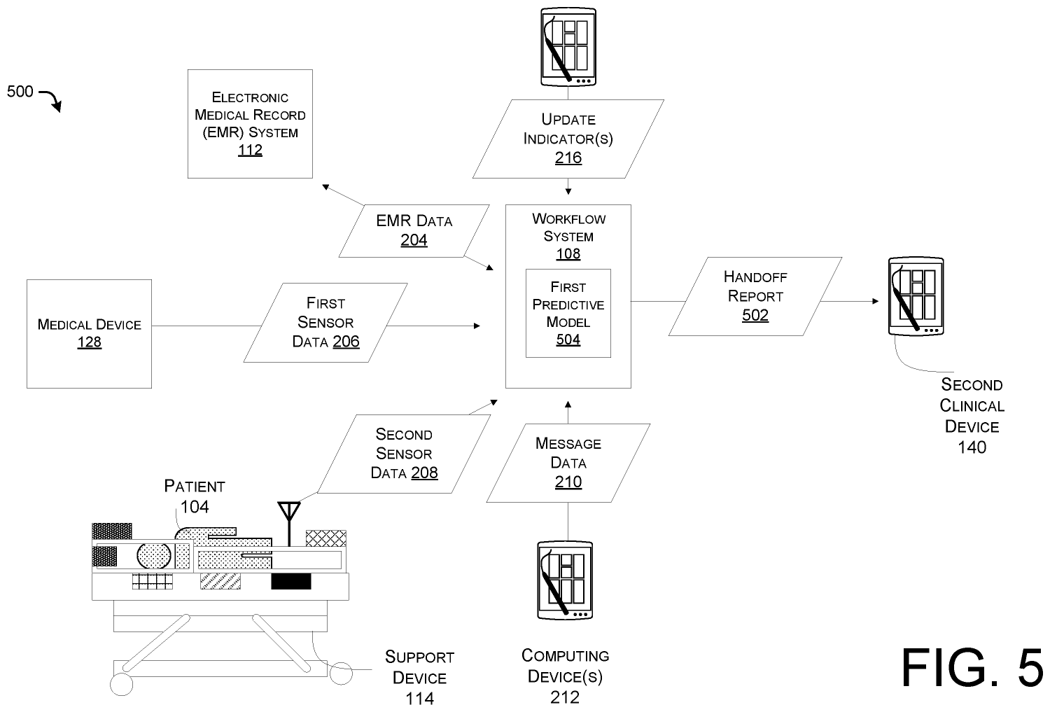

FIG. 5 illustrates example signaling 500 for providing a handoff report 502 to the second clinical device 140. As shown, the signaling 500 is between the first clinical device 106, the workflow system 108, the EMR system 112, the support device 114, the medical device 128, and the RTLS sensors 134 described above with respect to FIG. 1. Further, the signaling 500 involves the EMR data 204, the first sensor data 206, the second sensor data 206, the message data 210, the computing device(s) 212, and the update indicator 216 described above with respect to FIG. 2. For example, care of the patient 104 is being handed off from a first care provider associated with the first clinical device 106 to a second care provider associated with the second clinical device 140.

The workflow system 108 may generate the handoff report 502 based on patient data, which may be obtained from a variety of sources. For example, the workflow system 108 may generate the handoff report 502 based, at least in part, on the EMR data 204 from the EMR system 112.

The workflow system 108 may generate the handoff report 502 based, at least in part, on the first sensor data 206 and the second sensor data 208. The first sensor data 206 and/or second sensor data 208 may be indicative of a real-time or previously observed condition of the patient 104.

The workflow system 108 may generate the handoff report 502 based, at least in part, on the message data 210 received from one or more computing devices 212. The message data 210 includes messages related to the patient 104, such as messages transmitted or generated while the first care provider was caring for the patient 104.

In various examples, the workflow system 108 may generate the handoff report 502 based, at least in part, on the update indicator(s) 216 received from the first clinical device 106. For example, the update indicator(s) 216 may indicate that a particular task (e.g., a toileting task) has been completed for the patient 104, and the workflow system 108 may generate the handoff report 502 based, at least in part, on the completion of the particular task.

According to some implementations, the workflow system 108 includes a first predictive model 504 configured to generate the handoff report 502 based on the EMR data 204, the first sensor data 206, the second sensor data 208, the message data 210, the update indicator(s) 216, or any combination thereof. The predictive model 504 may use retrospective, descriptive, predictive and prescriptive analytics to generate the handoff report 502. In particular, the first predictive model 504 may be configured to identify data within the EMR data 204, the first sensor data 206, the second sensor data 208, the message data 210, the update indicator(s) 216, or any combination thereof, that is associated with a condition of the patient 104 that has a (greater than a threshold) likelihood of occurring when the second care provider is caring for the patient 104. The first predictive model 504 may be configured to indicate the condition of the patient 104 in the handoff report 502. In some examples, the condition would be harmful to the patient 104, and the first predictive model 504 may identify one or more tasks that the second care provider can perform in order to reduce the likelihood that the condition will occur. The handoff report 502, for example, may indicate the task(s).

In some examples, the workflow system 108 may use preconfigured templates to generate the handoff report 502. For example, a champion care facility and/or champion care provider may be associated with good health outcomes for patients (e.g., as indicated in EMRs of patients cared for by the champion care facility and/or champion care providers). The workflow system 108 may generate a template by learning an arrangement and/or content of handoff reports utilized by the champion care facility and/or champion care provider. In some cases, the workflow system 108 may generate the handoff report 502 based on the template, such that other care facilities and care providers can benefit from the optimized handoff procedures implemented by the champion care facility and/or champion care provider.

The workflow system 108 may utilize various other techniques for generating the handoff report 502. In some cases, the workflow system 108 may use retrospective learning in order to determine the relevance of a piece of clinical information (e.g., EMR data, sensor data, and/or messaging data) and to know its relevance in generating the handoff report 502. For example, the workflow system 108 may use retrospective analytics to learn how to summarize clinical information in the handoff report 502. In some examples, the workflow system 108 may utilize predictive algorithms in order to understand a position of the patient 104 in a care pathway, and to communicate the next best tasks and their optimal sequencing in the handoff report 502. In some examples, the workflow system 108 computes optimal priortization multiple patients including the patient 104 and tasks and indicates them in the handoff report 502. For example, the workflow system 108 uses prescriptive analytics to generate the handoff report 502.

In various examples, the first predictive model 504 includes a machine learning model that is trained to identify the condition and the task(s). For example, the first predictive model 504 includes a support vector machine, a neural network, a deep learning neural network (e.g., a convolutional neural network), a decision tree, a random forest, deep learning neural network, a regression model (e.g., a logistic regression model, a polynomial regression model, etc.), a Bayesian network, or a combination thereof. In various examples, the first predictive model 504 includes one or more parameters that are optimized based on training data. The training data indicates previously observed patient data associated with the patient 104 or other patients and conditions that the patient 104 or other patients developed after the patient data was detected or otherwise obtained. In some cases, the previously observed data includes EMR data (e.g., including the EMR data 204), sensor data obtained from the medical device 128 or other medical devices (e.g., including the first sensor data 206), sensor data obtained from the support device 114 or other support devices (e.g., including the second sensor data 208), message data obtained from the computing device(s) 212 (e.g., the message data 110), or other patient data associated with the patient 104 or other patients during a time interval that occurs prior to the second care provider taking over care of the patient 104. For example, the parameter(s) are optimized based on correlations between the patient data and the conditions of the patient.

In particular examples, the first predictive model 504 is trained to identify that the patient 104 is at risk of developing sepsis while the second care provider is scheduled to take care of the patient 104. For instance, training data including patient data of previous patients may have indicated a correlation between individuals that received a pacemaker from a particular manufacturer, an average heart rate of greater than 100 beats per minute during a particular time interval (e.g., one hour), and a particular trend of body temperature (e.g., a body temperature that ranges between 35 and 38 degrees Celsius over the course of an hour) and a heightened risk of developing sepsis within three hours. The first predictive model 504 is configured to identify this correlation based on the training data. The first predictive model 504 may be configured identify that the patient 104 has the heightened risk of developing sepsis because the EMR data 204, the first sensor data 206, and the second sensor data 208 indicate that the patient 104 has received a pacemaker from the particular manufacturer, has had an average heart rate of greater than 100 beats per minute during a particular hour, and has had a body temperature that has dipped below 35 degrees Celsius and has risen above 38 degrees Celsius during the particular hour. Accordingly, the workflow system 108 may generate the handoff report 502 to indicate that the patient 104 is at risk for developing sepsis and include a recommendation that the patient 104 receive antibiotics within the first hour of the second care provider taking over care of the patient 104.

In various implementations, the workflow system 108 may indicate at least a portion of the patient data of the patient 104 that was obtained prior to the second care provider taking over care of the patient 104. In some cases, the first predictive model 504 is configured to identify a portion of the patient data that is most relevant to continuing care of the patient 104. For example, if the medical device 128 includes a blood pressure sensor and detects that a blood pressure of the patient 104 is in a healthy physiological range, the workflow system 108 may refrain from including the blood pressure of the patient 104 in the handoff report 502. However, if the blood pressure of the patient 104 is outside of a healthy physiological range, the workflow system 108 may indicate the blood pressure of the patient 104 in the handoff report 502. Thus, the handoff report may omit extraneous information that is not or minimally relevant to the care to be provided by the second care provider.

In various examples, the handoff report 502 may indicate a status of the patient 104 as well as one or more clinical tasks to be performed for the patient 104. In some cases, the handoff report 502 may indicate statuses of multiple patients (including the patient 104) and tasks associated with the multiple patients. In various implementations, the workflow system 108 and/or the first predictive model 504 generates the handoff report 502 to include only the most relevant information associated to the care of the multiple patients, such that the second care provider is able to prioritize the care of the multiple patients during the time that the second care provider is assigned to care for the multiple patients.

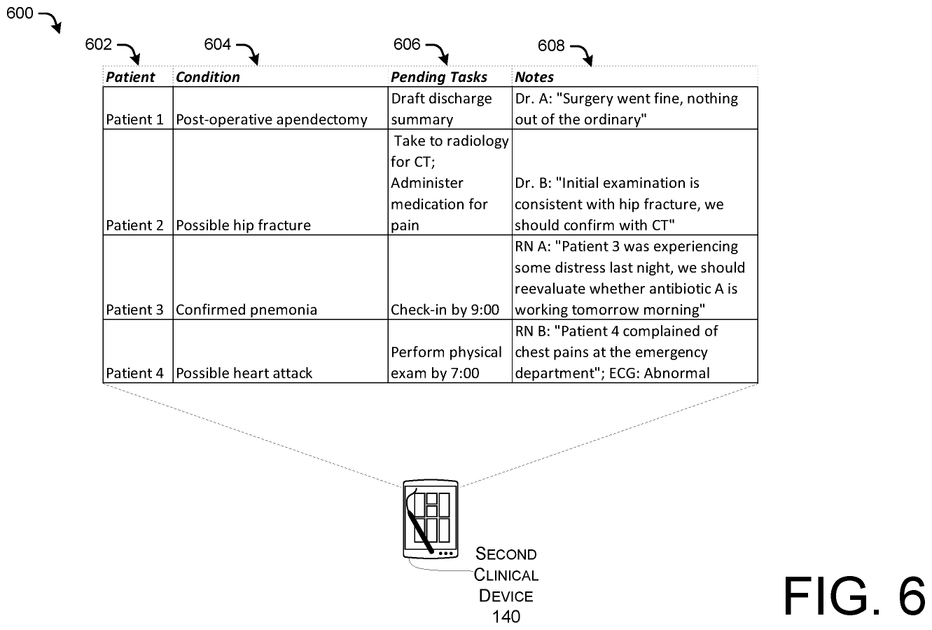

FIG. 6 illustrates an example GUI 600 of a handoff report output by the second clinical device 140. The second care provider associated with the second clinical device 140 is assigned four patients: Patient 1 to Patient 4. The example GUI 600 includes a table, for instance. The table includes a patient 602 field, a condition 604 field, a pending tasks 606 field, and a notes 608 field. The patient 602 field may indicate an identifier of Patient 1 through Patient 4, such as names, locations, ID numbers, or other identifying information.

The condition 604 field indicates a condition of each patient. In some cases, the conditions are confirmed. For instance, Patient 1 is “post-operative appendectomy” and Patient 3 has “confirmed pneumonia.” In some cases, the confirmed conditions are indicated directly in the EMRs of the patients. Some of the conditions may be suspected. For example, Patient 2 has a “possible hip fracture” and Patient 4 has a “possible heart attack.” In various examples, a computing model is configured to identify the suspected conditions based on patient data of Patient 1 to Patient 4.

The pending tasks 606 field summarizes one or more tasks to be performed by the care provider for each patient. In some examples, the tasks are generated by a computing model based on the patient data associated with the patients. For example, Patient 2 has a “possible hip fracture.” The computing model may determine that the best course of care for patients with possible hip fractures includes imaging and pain treatment. Accordingly, the computing model may indicate tasks including “take to radiology for CT” and “administer medication for pain.”

The notes 608 field summarizes a portion of patient data relevant to the care of the patients. In some cases, the notes 608 field indicates message data and/or EMR data indicating a message provided by another care provider. For instance, the notes 608 field for Patient 1 indicates that Dr. A has noted that the “surgery went fine, nothing out of the ordinary,” indicating that Patient 1 is unlikely to require more than normal oversight by the second care provider. In some examples, the notes 608 field indicates sensor data. For example, the notes 608 field for Patient 4 indicates a message from RN B as well as an indication that an ECG of the Patient 4 is abnormal.

In some examples, the GUI 600 of the handoff report is optimized based on the care provider. For example, if the care provider prefers to not receive recommendations for tasks, the care provider can modify the GUI 600 to omit the pending tasks 306 field. In some examples, the care provider may prefer to view additional information on the handoff report. For example, the care provider may prefer to view a field indicating sepsis risks of Patient 1 to Patient 4. In some implementations, another computing model learns and optimizes the GUI 600 based on interactions between the care provider and the GUI 600 or previous handoff reports provided to the care provider.

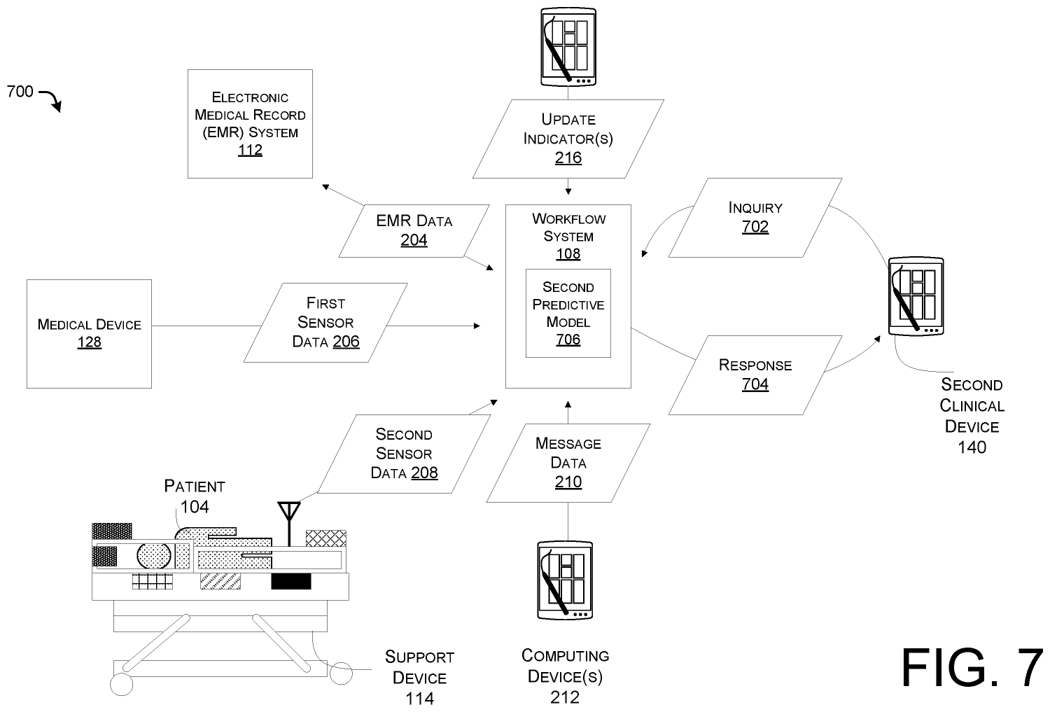

FIG. 7 illustrates example signaling 700 for optimizing a handoff report for a particular care provider. As shown, the signaling 700 is between the first clinical device 106, the workflow system 108, the EMR system 112, the support device 114, the medical device 128, and the second clinical device 140 described above with reference to FIG. 1. Further, the signaling 700 involves the EMR data 204, the first sensor data 206, the second sensor data 206, the message data 210, the computing device(s) 212, and the update indicator 216 described above with respect to FIG. 2.

In FIG. 7, the workflow system 108 has provided a first handoff report (e.g., the handoff report 502) to the second clinical device 140. However, the first handoff report may be unsatisfactory to the particular care provider. For example, the particular care provider may request additional information about one or more of the patients whose conditions are summarized on the first handoff report.

In various implementations, the workflow system 108 may act as a personal clinical assistant for the particular care provider. As used herein, the term “personal clinical assistant,” and its equivalents, can refer to hardware and/or software configured to provide on-demand information regarding the care of a patient. The second clinical device 140, for example, may transmit an inquiry 702 to the workflow system 108 based on an input signal from the particular care provider. The inquiry 702 may be a request for additional information or less information about a particular patient.

The workflow system 108 may generate a response 704 to the inquiry 702. In various examples, the workflow system 108 generates the response 704 based on patient data, such as the EMR data 204, the first sensor data 206, the second sensor data 208, the message data 210, the update indicator(s) 216, or a combination thereof. The workflow system 108 transmits the response 704 to the second clinical device 140. In some cases, the response 704 is an update to the handoff report provided to the second clinical device 140.

In a particular example, the handoff report initially omits a heart rate of the patient 104. However, the particular care provider would like to review the heart rate of the patient 104 prior to starting his or her shift. The particular care provider may speak, into a microphone of the second clinical device 140, the phrase “what is the heart rate of patient 104?” The second clinical device 140 generates the inquiry 702 to include an audio signal based on the phrase detected by the microphone. The workflow system 108 may perform natural language processing on the audio signal in order to determine that the particular care provider is requesting the heart rate of patient 104. The medical device 128, for example, is configured to detect the heart rate of the patient 104 (automatically or on-demand by the workflow system 108) and transmit an indication of the heart rate to the workflow system 108 in the first sensor data 206. The workflow system 108 may generate the response 704 to include the heart rate. Thus, the workflow system 108 may provide requested information to the second clinical device 140 on-demand.

In addition, the workflow system 108 includes a second predictive model 706 configured to identify a template of a second handoff report to provide to the second clinical device 140. The second predictive model 706 may use retrospective, descriptive, predictive and prescriptive analytics to generate the response 704. The template includes one or more components. As used herein, the term “components,” and its equivalents, refers to information and/or user interface elements included in the handoff report. The second predictive model 706, for instance, includes a support vector machine, a neural network, a deep learning neural network (e.g., a convolutional neural network), a decision tree, a random forest, deep learning neural network, a regression model (e.g., a logistic regression model, a polynomial regression model, etc.), a Bayesian network, or a combination thereof. In various examples, the second predictive model 706 includes one or more parameters that are optimized based on training data. The training data may include the inquiry 702 and the response 704. Thus, the second predictive model 706 adapts the template based on the inquiry 702 and the response 704. For example, the second predictive model 706 may learn, based on the inquiry 702, that the particular care provider would prefer to identify the heart rates of patients in the second handoff report, and may therefore adapt the template to include a component corresponding to the heart rates.

In various implementations, the workflow system 108 stores the template in a template database. The template database, for instance, includes multiple handoff report templates associated with various care providers. Thus, upon identifying that the particular care provider will be taking over care of one or more patients, the workflow system 108 may access the stored template associated with the particular care provider and generate the second handoff report with minimal delays.

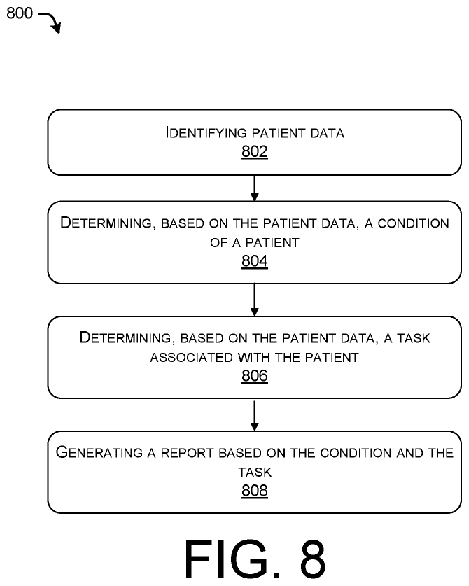

FIG. 8 illustrates an example process 800 for assisting a care provider by providing a report summarizing current and/or potential conditions of a patient assigned to the care provider. In some cases, the report summarizes the current and/or potential conditions of multiple patients assigned to the care provider, but FIG. 8 will be described with respect to a single patient for the sake of simplicity. The process 800 is performed by an entity, such as a computing system, one or more processors in the computing system, the first clinical device 106, the workflow system 108, the EMR system 112, the support device 114, the medical device 128, or the second clinical device 140 described above.

At 802, the entity identifies patient data. In various examples, the patient data is indicative of a condition of a patient assigned to a care provider. For example, the patient data indicates one or more parameters of the patient. In some cases, the patient data includes at least one of a medication prescribed and/or administered to the patient, a time at which the medication was prescribed and/or administered to the patient, a vital sign of the patient, a medical history of the patient, a time at which the vital sign was detected, a sepsis risk of the patient, a procedure performed on the patient, or a time at which the procedure was performed on the patient.

The patient data may include sensor data, EMR data, message data, or a combination thereof. The sensor data, for instance, includes data obtained from a medical device and/or support device associated with the patient. In some examples, the sensor data includes at least one image and/or video of the patient. In some cases, the sensor data includes a parameter (e.g., a vital sign) of the patient detected by the medical device and/or the support device. In various implementations, the EMR data includes data stored in an EMR of the patient. For example, the EMR of the patient is stored in an external server that is in communication with the entity. The message data, for example, includes a text, audio, or video message about the patient. For example, the message may be transferred between computing devices associated with the patient, one or more care providers, or one or more family members of the patient.

At 804, the entity determines, based on the patient data, a condition of a patient. For example, the condition includes at least one of a BMAT of the patient, a fall risk of the patient, a sepsis risk of the patient, or an availability of the patient for receiving a visit from the care provider. In some examples, the entity predicts a condition of the patient. For example, the entity uses the patient data to predict a risk that the patient will develop sepsis within the next 24 hours.

At 806, the entity determines, based on the patient data, a task associated with the patient. The task, for example, can be a diagnostic activity to be performed on the patient (e.g., a vitals sign check, medical imaging, etc.), a therapeutic activity to be performed on the patient (e.g., administration of a medication, physical therapy, a surgery, etc.), an activity to prevent medical complications (e.g., toileting assistance, sheet change, replacement of a port or other invasive and/or semi-invasive device, etc.), and so on. In some examples, the task is determined based on the condition of the patient. For example, the task may be associated with the BMAT of the patient (e.g., a task to move the patient in accordance with the BMAT), the fall risk of the patient (e.g., a task to reduce the fall risk of the patient), or a sepsis risk of the patient (e.g., a task to reduce the sepsis risk of the patient). According to some examples, the entity further determines whether the task is associated with equipment. In some cases, the entity uses RTLS techniques to determine whether the equipment is located within a vicinity (e.g., the room and/or within a threshold distance) of the patient.

At 808, the entity generates a report based on the condition and the task. In various examples, the entity provides the report to a clinical device associated with the care provider. In some cases, the report is a workflow report or a handoff report. For example, the entity may transmit the report to the clinical device within a threshold time period of the patient being handed over to the care provider. The report may summarize the condition and the task. For example, the report may indicate whether the patient is available for a visit from the care provider, whether the equipment is located within the vicinity of the patient, and so on. The clinical device may output the report to the care provider. In some examples, the report is generated based on a template associated with the care provider, such that the report includes information that the entity has learned may be relevant to the care provider.

In some examples, the entity modifies the report or provides additional information to the care provider based on messages transmitted from the clinical device to the entity after the report is output to the care provider. For example, the care provider can input an inquiry about the condition of a patient into the clinical device, which may transmit a signal indicative of the inquiry to the entity. In some cases, the entity may perform natural language processing and/or pattern recognition on the signal to process the inquiry and may generate a response based on the patient data. In some examples, the care provider may input, into the clinical device, an update indicating that the condition of the patient has changed or that the task has been completed. The clinical device may transmit a signal indicative of the update to the entity, which may update the report based on the update. In some examples, the entity may update the EMR of the patient based on the update.

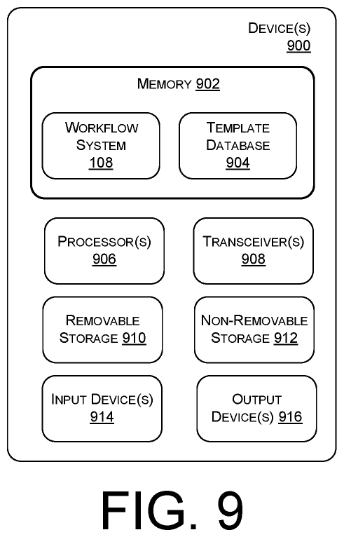

FIG. 9 illustrates at least one example device configured to enable and/or perform the some or all of the functionality discussed herein. The device(s) 900 can be implemented as one or more server computers, a network element on a dedicated hardware, as a software instance running on a dedicated hardware, or as a virtualized function instantiated on an appropriate platform, such as a cloud infrastructure, and the like. It is to be understood in the context of this disclosure that the device(s) 900 can be implemented as a single device or as a plurality of devices with components and data distributed among them.

As illustrated, the device(s) 900 includes a memory 902. In various embodiments, the memory 902 is volatile (including a component such as Random Access Memory (RAM)), non-volatile (including a component such as Read Only Memory (ROM), flash memory, etc.) or some combination of the two. The memory 902 may include various components, such as the workflow system 108 and/or a template database 904, and other components. These components can include methods, threads, processes, applications, or any other sort of executable instructions. The workflow system 108, the template database 904, and various other elements stored in the memory 902 can also include files and databases.

The memory 902 may include various instructions (e.g., instructions in the workflow system 108 and/or template database 904), which can be executed by at least one processor 906 to perform operations. In some embodiments, the processor(s) 906 includes a Central Processing Unit (CPU), a Graphics Processing Unit (GPU), or both CPU and GPU, or other processing unit or component known in the art. In some examples, the processor(s) 906 include chipsets configured to perform complex (e.g., AI-based) computations in the vicinity of sensors, such as chipsets designed for Internet-of-Things (IoT)-based architectures.

The device(s) 900 can also include additional data storage devices (removable and/or non-removable) such as, for example, magnetic disks, optical disks, or tape. Such additional storage is illustrated in FIG. 9 by removable storage 910 and non-removable storage 912. Tangible computer-readable media can include volatile and nonvolatile, removable and non-removable media implemented in any method or technology for storage of information, such as computer readable instructions, data structures, program modules, or other data. The memory 902, removable storage 910, and non-removable storage 912 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 900. Any such tangible computer-readable media can be part of the device(s) 900.

The device(s) 900 also can include input device(s) 914, such as a keypad, a cursor control, a touch-sensitive display, voice input device, etc., and output device(s) 916 such as a display, speakers, printers, etc. These devices are well known in the art and need not be discussed at length here. In particular implementations, a user can provide input to the device(s) 900 via a user interface associated with the input device(s) 914 and/or the output device(s) 916.

As illustrated in FIG. 9, the device(s) 900 can also include one or more wired or wireless transceiver(s) 908. For example, the transceiver(s) 908 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 908 can utilize Multiple-Input/Multiple-Output (MIMO) technology. The transceiver(s) 908 can include any sort of wireless transceivers capable of engaging in wireless, Radio Frequency (RF) communication. The transceiver(s) 908 can also include other wireless modems, such as a modem for engaging in Wi-Fi, WiMAX, Bluetooth, or infrared communication.

### Example Clauses

1. A workflow system, including: at least one processor; and memory storing a template database and instructions that, when executed by the at least one processor, cause the workflow system to perform operations including: determining that care of a patient is being transferred from a first care provider to a second care provider at a particular time; retrieving, from the template database, a template associated with the second care provider; identifying patient data including: message data that includes at least one text, audio, or video message about the patient that is transmitted to or from a first computing device associated with the first care provider, electronic medical record (EMR) data that includes at least a portion of an EMR of the patient, and sensor data that includes a physiological parameter of the patient detected by a hospital bed on which the patient is supported; generating a handoff report based on the template and the patient data, the handoff report including a graphical user interface (GUI) summarizing a predicted condition of the patient and at least one task associated with care of the patient; and within a threshold time period of the particular time, transmitting, to a second computing device associated with the second care provider, the handoff report.
2. The workflow system of clause 1, wherein the operations further include: determining, by a computing model, the predicted condition of the patient based on the patient data, the predicted condition including sepsis, a pressure injury, choking, vomiting, breathing problems, apnea, delirium, insufficient sleep quality, cardiac shock, organ failure, immobility, a health deterioration, or a fall of the patient; and determining, by the computing model, the at least one task associated with preventing the sepsis or the fall of the patient.
3. The workflow system of clause 2, the patient being a first patient, the message data being first message data, the EMR data being first EMR data, the sensor data being first sensor data, and the hospital bed being a first hospital bed, wherein the computing model includes a machine learning model, and wherein the operations further include: identifying training data including: second message data that includes messages about second patients that are transmitted to or from third computing devices associated with third care providers, second EMR data that includes at least portions of EMRs of the second patients, second sensor data that includes physiological parameters of the second patients detected by second hospital beds, outcome data indicating conditions of the second patients, and task data indicating tasks performed on the second patients by the third care providers; and training the machine learning model to identify correlations between the second message data, the second EMR data, the second sensor data, the outcome data, and the task data.
4. The workflow system of any one of clauses 1 to 3, wherein the operations further include: receiving, from the second computing device, an inquiry about the patient; generating a response to the inquiry based on the patient data; transmitting, to the second computing device, the response; modifying the template by training a machine learning model based on the inquiry and the response; and based on modifying the template, storing the template in the template database.
5. The workflow system any one of clauses 1 to 4, wherein the sensor data further includes: at least one image of the patient that has been captured by a camera mounted on the hospital bed, a cart, a ceiling, or a wall; and audio of the patient that has been recorded by a microphone mounted on the hospital bed, the cart, the ceiling, or the wall.
6. The workflow system of any one of clauses 1 to 5, the physiological parameter being a first physiological parameter, wherein the sensor data further includes a second physiological parameter of the patient that has been detected by a medical device external to the hospital bed.
7. A workflow system, including: at least one processor; and memory storing instructions that, when executed by the at least processor, cause the at least one processor to perform operations including: identifying patient data including: message data that includes at least one text, audio, or video message about a patient, electronic medical record (EMR) data that includes at least a portion of an EMR of the patient, and sensor data that includes at least one image the patient detected by a camera mounted on a hospital bed on which the patient is supported; determining, based on the patient data, a condition of the patient; determining, based on the patient data, a task associated with the patient to be performed by a care provider; generating a report including the condition of the patient and the task; and transmitting, to a computing device associated with the care provider, the report.
8. The workflow system of clause 7, wherein the patient data includes at least one of a medication prescribed and/or administered to the patient, a time at which the medication was prescribed and/or administered to the patient, a vital sign of the patient, a medical history of the patient, a time at which the vital sign was detected, a sepsis risk of the patient, a falls risk of the patient, a pressure injury risk of the patient, a procedure performed on the patient, or a time at which the procedure was performed on the patient.
9. The workflow system of clause 7 or 8, wherein the condition includes at least one of a mobility score of the patient, a fall risk of the patient, a sepsis risk of the patient, or an availability of the patient for receiving a visit from the care provider.
10. The workflow system of any one of clauses 7 to 9, wherein the operations further include: receiving, from the computing device, an update message indicating that the task has been completed, and updating the report based on the update message.
11. The workflow system of any one of clauses 7 to 10, wherein the operations further include: receiving, from the computing device, an update message indicating that the condition of the patient has changed or that the task has been completed; and updating the EMR of the patient based on the update message.
12. The workflow system of any one of clauses 7 to 11, wherein the operations further include: determining that completion of the task is associated with equipment; determining a location of the equipment; and determining that the location of the equipment is in a room associated with the patient or is within a threshold distance of the patient, and wherein the report further includes an indication that the location of the equipment is in the room associated with the patient or is within the threshold distance of the patient.
13. The workflow system of any one of clauses 7 to 12, wherein the operations further include: determining, based on the sensor data, that the patient is unavailable for a visit from the care provider during a time period, wherein the report further includes an indication that the patient is unavailable for the visit from the care provider during the time period.
14. The workflow system of any one of clauses 7 to 13, wherein the operations further include: determining that care of the patient is being handed off to the care provider at a time, and wherein transmitting the report is performed within a threshold time period of the time.
15. The workflow system of any one of clauses 7 to 14, wherein the operations further include: determining, based on the patient data, whether care of the patient is compliant with a care guideline, the care guideline specifying a goal timing of performing a procedure on the patient; and outputting, to a computing device associated with an administrator of a care facility in which the patient is admitted, a compliance report indicating whether the care of the patient is compliant with the care guideline.
16. A workflow system, including: at least one processor; and memory storing instructions that, when executed by the at least processor, cause the at least one processor to perform operations including: identifying patient data including: message data that includes at least one text, audio, or video message about multiple patients assigned to a care provider, electronic medical record (EMR) data that includes at least a portion of EMRs of the multiple patients, and sensor data detected by support structures and medical devices monitoring the multiple patients; determining, based on the patient data, conditions of the multiple patient; determining, based on the patient data, availability statuses of the multiple patients for visits from the care provider; determining, based on the patient data, tasks associated with the patients to be performed by the care provider; generating a workflow report including the conditions of the multiple patients, the availability statuses of the multiple patients, and the tasks; and transmitting, to a computing device associated with the care provider, the report.
17. The workflow system of clause 16, wherein the sensor data includes at least one image of a particular patient among the multiple patients, and wherein determining the availability statuses of the multiple patients includes: determining, based on the at least one image, that the particular patient is sleeping, in an appointment with another care provider, or is outside of a room associated with the particular patient.
18. The workflow system of clause 16 or 17, wherein the multiple patients include a particular patient, and wherein determining the availability statuses of the multiple patients includes: predicting, based on the sensor data, that the particular patient will be sleeping during a time period, and wherein the workflow report indicates that the particular patient is predicted to be unavailable during the time period.
19. The workflow system of clause 18, the particular patient being a first patient, the multiple patients further including a second patient, wherein the operations further include: determining that the second patient is available during the time period, wherein the workflow report indicates that the second patient is available during the time period.
20. The workflow system of any one of clauses 16 to 19, wherein the conditions of the multiple patients include mobility scores of the multiple patients, sepsis risks of the multiple patients, and fall risks of the multiple patients.
21. The workflow system of any one of clauses 16 to 20, wherein the operations further include: receiving, from the computing device, an update message indicating that at least one of the tasks have been completed, and updating the report based on the update message.
22. The workflow system of any one of clauses 16 to 21, wherein the operations further include: determining that completion of a particular task among the tasks is associated with equipment; determining a location of the equipment; and determining that the location of the equipment is in a room associated with a patient corresponding to the particular task or is within a threshold distance of the patient, and wherein the report further includes an indication that the location of the equipment is in the room associated with the patient or is within the threshold distance of the patient.
23. The workflow system of any one of clauses 16 to 22, wherein the operations further include: determining that the first user and the second user are being handed off to the provider at a first time, and wherein transmitting the message is performed at second time, the second time being within a threshold time period of the first time.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

1. A system, comprising:
at least one processor; and
memory storing instructions that, when executed by the at least one processor, cause the system to perform operations comprising:
determining, based on an event, that care of a user is being transferred from a first provider to a second provider at a particular time;
retrieving, via a network, a template associated with the second provider;
identifying user data comprising:
first data that comprises at least one text, audio, or video message about the user that is transmitted to or from a first computing device associated with the first provider,
second data that comprises at least a portion of a record of the user, and
third data that comprises a physiological parameter of the user detected by a device on which the user is supported;
generating a handoff message based on the template and the user data, the handoff message comprising a graphical user interface (GUI) summarizing a condition of the user and at least one task; and
within a threshold time period of the particular time, transmitting, to a second computing device associated with the second provider, the handoff message.

2. The system of claim 1, wherein the operations further comprise:
determining, by a computing model, the condition of the user based on the user data, the condition comprising sepsis, a pressure injury, choking, vomiting, breathing problems, apnea, delirium, insufficient sleep quality, cardiac shock, organ failure, immobility, a deterioration, or a fall of the user; and
determining, by the computing model, the at least one task associated with preventing the sepsis or the fall of the user.

3. The system of claim 2, the user being a first user, wherein the computing model comprises a machine learning model, and
wherein the operations further comprise:
identifying training data comprising:
fourth data that comprises messages about second users that are transmitted to or from third computing devices associated with third providers,
fifth data that comprises at least portions of records of second users,
sixth data that comprises physiological parameters of the second users detected by second devices,
seventh data indicating conditions of the second users, and
eighth data indicating tasks performed on the second users by the third providers; and
training the machine learning model to identify correlations between the fourth data, the fifth data, the sixth data, the seventh data, and the eighth data.

4. The system of claim 1, wherein the operations further comprise:
receiving, from the second computing device, an inquiry about the user;
generating a response to the inquiry based on the user data;
transmitting, to the second computing device, the response;
modifying the template by training a machine learning model based on the inquiry and the response; and
based on modifying the template, storing the template in a database.

5. The system of claim 1, wherein the third data further comprises:
at least one image of the user that has been captured by a camera mounted on the device, a cart, a ceiling, or a wall; and
audio of the user that has been recorded by a microphone mounted on the device, the cart, the ceiling, or the wall.

6. The system of claim 1, the physiological parameter being a first physiological parameter, wherein the third data further comprises a second physiological parameter of the user that has been detected by a second device external to the device.

7. A system, comprising:
at least one processor;
a digital camera operably connected to the at least one processor and having a field of view, the digital camera being positioned such that a device, on which a user is disposed, is disposed within the field of view; and
memory storing instructions that, when executed by the at least processor, cause the at least one processor to perform operations comprising:
identifying user data comprising:
first data that comprises at least one text, audio, or video message about the user,
second data that comprises at least a portion of a record of the patient, and
third data that comprises at least one image of the user captured by the camera while the user is disposed on the device;
determining, based on the user data, a condition of the user;
determining, based on the user data, an action associated with the user to be performed by a provider;
generating a message comprising the condition of the user and the action; and
transmitting, to a computing device associated with the provider, the message.

8. The system of claim 7, wherein the condition of the user comprises a BMAT of the user, sepsis risk of the user, and fall risk of the user.

9. The system of claim 7, wherein the third data comprises at least one image of the first user, and the operations further comprise:
determining a status of the user based at least in part on determining, based on the at least one image, that the user is sleeping, in an appointment with another provider, or is outside of a room associated with the user.

10. The system of claim 7, wherein the operations further comprise:
determining a status of the user based on predicting, based on the third data, that the user will be sleeping during a time period, and
wherein the message indicates that the user is predicted to be unavailable during the time period.

11. The system of claim 7, wherein the operations further comprise:
receiving, from the computing device, an update message indicating that the action has been completed, and
updating the second data of the user based on the update message.

12. The system of claim 7, wherein the operations further comprise:
receiving, from the computing device, an update message indicating that the condition of the user has changed or that the action has been completed; and
updating the second data of the user based on the update message.

13. The system of claim 7, wherein the operations further comprise:
determining that completion of the action is associated with equipment;
determining a location of the equipment; and
determining that the location of the equipment is in a room associated with the user or is within a threshold distance of the user, and
wherein the message further comprises an indication that the location of the equipment is in the room associated with the user or is within the threshold distance of the user.

14. The system of claim 7, wherein the operations further comprise:
determining, based on the third data, that the user is available during a time period,
wherein the message further comprises an indication that the user is available during the time period.

15. The system of claim 7, wherein the operations further comprise:
determining that the user is being handed off to the provider at a first time, and
wherein transmitting the message is performed at second time, the second time being within a threshold time period of the first time.

16. A system, comprising:
at least one processor; and
memory storing instructions that, when executed by the at least one processor, cause the at least one processor to perform operations comprising:
identifying user data comprising:
first data that comprises at least one text, audio, or video message about a first user and a second user assigned to a provider,
second data that comprises at least a portion of a first record of the first user and a portion of a second record of the second user, and
third data captured by sensors associated with support structures and devices monitoring the first user and the second user;
determining, based on the user data, a first condition of the first user and a second condition of the second user;
determining, based on the user data, a first status of the first user and a second status of the second user, the first status and the second status indicating availability of the first user and the second user for visits from the provider;
determining, based on the user data, actions associated with the first user and the second user to be performed by the provider;
generating a message comprising the first condition of the first user, the second condition of the second user, the first status of the first user, the second status of the second user, and the actions; and
transmitting, to a computing device associated with the provider, the message.

17. The system of claim 16, wherein the third data comprises at least one image of the first user, and
wherein determining the first status of the first user comprises:
determining, based on the at least one image, that the first user is sleeping, in an appointment with another provider, or is outside of a room associated with the first user.

18. The system of claim 16,
wherein determining the first status of the first user comprises:
predicting, based on the third data, that the first user will be sleeping during a time period, and
wherein the message indicates that the first user is predicted to be unavailable during the time period.

19. The system of claim 18, wherein the operations further comprise:
determining that the second user is available during the time period,
wherein the message indicates that the second user is available during the time period.

20. The system of claim 16, wherein the first condition of the first user comprises a BMAT of the first user, sepsis risk of the first user, and fall risk of the first user.

21. The system of claim 16, wherein the operations further comprise:
receiving, from the computing device, an update message indicating that at least one of the actions have been completed, and
updating the message based on the update message.

22. The system of claim 16, wherein the operations further comprise:
determining that completion of an action is associated with equipment;
determining a location of the equipment; and
determining that the location of the equipment is in a room associated with the first user or the second user or is within a threshold distance of the first user or the second user, and
wherein the message further comprises an indication that the location of the equipment is in the room associated with the first user or the second user or is within the threshold distance of the first user or the second user.

23. The system of claim 16, wherein the operations further comprise:
determining that the first user and the second user are being handed off to the provider at a first time, and
wherein transmitting the message is performed at second time, the second time being within a threshold time period of the first time.
